(19) United States
(12) Patent Application Publication
(10) Pub. No.: US 2024/0145053 A1
(43) Pub. Date: May 2, 2024

(54) COMPUTER-IMPLEMENTED SYSTEMS AND METHODS FOR PERSISTENT HEALTH DATA COLLECTION AND MULTI-LEVEL PRIORITIZATION

(71) Applicant: JuvYou (Europe) Limited, Dublin 2 (IE)

(72) Inventors: Joshua ANTHONY, Princeton Junction, NJ (US); Amy KEELER, Conshohocken, PA (US); Samia NORPEL, Blue Bell, PA (US); Maurice ZAKI, Summit, NJ (US)

(73) Assignee: JuvYou (Europe) Limited, Dublin 2 (IE)

(21) Appl. No.: 18/497,861

(22) Filed: Oct. 30, 2023

Related U.S. Application Data
(60) Provisional application No. 63/381,743, filed on Oct. 31, 2022.

(51) Int. Cl. G16H 10/65; G16H 20/00; G16H 40/63

(52) U.S. Cl. CPC G16H 10/65; G16H 20/00; G16H 40/63

(57) ABSTRACT

Computer-implemented systems, methods, and computer-readable media are provided for persistent health data collection and multi-level prioritization. In accordance with one implementation, a method is provided that includes steps performed by at least one processor. The method may include providing at least one graphical user interface to a user that is configured to receive user input and creating, using application program interfaces (APIs), persistent connections to collect health data from one or more devices, wherein a first prioritization for the connected devices is stored based on user input received through the at least one graphical user interface. The method may further include storing a first prioritization for the data, the first prioritization for the data defining an order among the health data from the connected devices for selecting and applying one or more health data parameters, receiving health data from the devices in accordance with the first prioritization for the devices, and calculating a health value for the user using one more health data parameters based on the received health data, wherein the health data parameters are selected and applied according to the first prioritization for the data.

### TECHNICAL FIELD

The present disclosure relates generally to computer-implemented systems and methods for collecting, managing, and processing data, including health data of users. More specifically, and without limitation, this disclosure relates to computer-implemented systems and methods for persistent health data collection and multi-level prioritization. This disclosure also relates to the systematic analysis of prioritized data for users, and systems and methods for generating and providing personalized health values and health and/or wellness recommendations. The systems and methods disclosed herein may be used in various applications, including to monitor, manage, and improve health-related components of users such as metabolic health, cardiovascular health, muscle health, immune health, and psychological health, among others.

### BACKGROUND

Modern health analysis systems process data from personal medical records, tests, and other sources to analyze the health or wellness of users. With advancements in technology, an increasing number of personal electronic devices and software-based applications are now available to track a user's activity and health data (e.g., weight, heart rate, blood oxygen saturation (SpO2) level, breathing rate, respiratory rate, walk or activity level, calorie consumption, sleep patterns, stress levels, and so on). For example, a wide variety of health and fitness trackers are commercially available, including fitness tracking bands, rings, watches, apps, and other devices. However, there are challenges in terms of connectivity and techniques for collecting and managing health data. Also, there are challenges in terms of data availability and prioritization for health and wellness calculations.

Extant health analysis systems are often limited in terms of options, compatibility, and/or user-defined configurations for data connectivity, prioritization, and privacy. Some extant solutions may be limited in terms of what devices or sources may be used to provide health data or they may be self-contained or closed systems that collect data only from specific devices or sensors associated with the system. Extant solutions also suffer from other drawbacks such as limited capabilities in terms of data persistence, processing, and/or data sourcing and maintenance for health and wellness calculations. Such drawbacks and limitations can make extant health analysis systems unreliable and/or unattractive to users.

In view of the foregoing, there is a need for improvements in computer-implemented health data analysis and management systems and applications. For example, there is a need for more advanced systems that permit data connectivity across multiple devices and other sources of health and wellness data. There is also a need for flexible data prioritization and privacy controls and improved methods for health data collection, maintenance, and processing to provide more reliable calculations and recommendations to users.

Modern health analysis systems also fail to account for circumstances where health data from a personal electronic device is unobtainable or unavailable. Therefore, there is a need for improvements in computer-implemented health data collection and management systems and applications to allow, among other configurations, the use of the next available or prioritized health data when top prioritized health is not available. Embodiments of the present disclosure provide such improvements and can address one or more of the above-noted drawbacks or disadvantages of available solutions.

### SUMMARY

The present disclosure generally relates to systems, methods, and computer-readable media with instructions executable by a processor for collecting and prioritizing data from devices and other sources of health data. Embodiments of the present disclosure also relate to computer-implemented systems and methods for collecting and maintaining prioritized data for users and generating personalized health values and health and/or wellness recommendations. Systems and methods consistent with the disclosed embodiments herein may be used in various applications, including to monitor, manage, and improve health-related components of a user such as metabolic health, cardiovascular health, muscle health, immune health, and psychological health, among others.

In accordance with some embodiments, computer-implemented methods are provided for persistent health data collection and multi-level prioritization. Consistent with one implementation, a method is provided that includes a plurality of steps performed by at least one processor. For example, the method may include providing at least one graphical user interface to a user that is configured to receive user input for one or more of a device selection, a device prioritization, and a data prioritization, and creating, using application program interfaces (APIs), persistent connections to collect health data from one or more devices, wherein a first prioritization for the connected devices is stored based on user input received through the at least one graphical user interface, the first prioritization for the connected devices defining an order for collecting health data from the connected devices. The method may also include storing, based on further user input received through the at least one graphical user interface, a first prioritization for the data, the first prioritization for the data defining an order among the data from the connected devices for selecting and applying one or more health data parameters. Further, the method may include receiving, using the persistent connections, health data from the devices in accordance with the first prioritization for the devices and calculating a health value for the user using one or more health data parameters based on the received health data, wherein the health data parameters are selected and applied according to the first prioritization for the data. By way of example, the health value may be a biological age or metabolic scale of the user.

Implementations related to the above method may include one or more of the following features. For example, using the persistent connections, the health data may be collected periodically from the one or more connected devices in the order defined by the first prioritization for the connected devices. Additionally, or alternatively, the health data may be collected in response to a triggering event. The triggering event may cause the health data to be automatically collected from the one or more connected devices in the order defined by the first prioritization for the connected devices. Additionally, or alternatively, the triggering event may cause the health data to be collected by prompting the user to manually enter health data through the at least one graphical user interface. The triggering event may be defined based on at least one of a condition, event, or schedule. By way of example, the triggering event may be defined based on at least one of: a period of time since the last collection of health data, a request for a health value, a request for a health or wellness recommendation, an authentication of the user, a connection or reconnection of a device, a data synchronization event or schedule, or an expiry of one or more health data parameters.

According to still further aspects, the user input received through the at least one graphical user interface may include manually entered health data for a user. The manually entered health data may include one or more health parameters. Through the at least one graphical user interface, the user may also prioritize manually entered health data or one or more health data parameters. Additionally, the first prioritization for the data may define an order for selecting and applying health data parameters among the health data received from the connected devices and manually entered health data received from the user through the at least one graphical user interface.

The health data may include user data and health data parameters including one or more of age, gender, ethnicity, location, quality of life measures, dietary intake and preferences, weight, height, arm circumference, waist circumference, systolic blood pressure, diastolic blood pressure, total blood pressure, high-density lipoprotein (HDL) cholesterol, low-density lipoprotein (LDL) cholesterol, total cholesterol, oxidized LDL (oxLDL) levels, triglyceride levels, plasma glucose levels, plasma insulin or c-peptide levels, non-esterified fatty acids (NEFA) levels, blood pressure, activity level, moderate muscle strength, vigorous muscle strength, walking distance per time unit, body fat percentage, squat strength, pushup strength, knee pushup strength, plank strength, balance tests, Short Physical Performance Battery (SPPB) score, sleep patterns, blood oxygen saturation (SpO2) level, maximum rate of oxygen (VO2), physical activity patterns, calorie consumption, resting heart rate, breathing rate, respiratory rate, health diseases, family history of health diseases, pregnancy, breast feeding patterns, pregnancy complications, contraceptive practices, perceived stress level, immune markers, vitamin and mineral levels, food intolerance, allergies or allergen markers, microbiome composition and markers, and genotypic or epigenetic information.

According to further aspects, the first prioritization for the connected devices is defined based on an order in which connections to the devices are established. The method may further include storing a second prioritization for the connected devices in response to further user input received through the at least one graphical user interface, the second prioritization for the connected devices being different from the first prioritization for the connected devices that defines an order for collecting health data from the connected devices.

The method may further include storing a second prioritization for the data in response to further user input received through the at least one graphical user interface, the second prioritization for the data being different from the first prioritization for the data that defines an order among the data from the connected devices or manually entered data for selecting and applying one or more health data parameters.

In still further implementations, the method includes parsing the received health data to obtain the one or more health data parameters. The parsing of the received health data may include applying one or more extract, transform, load (ETL) operations to the received health data to obtain the one or more health data parameters. Additionally, the method may include applying the one or more health data parameters to generate a health or wellness recommendation for the user. The method may include applying one or more health data parameters to a health engine that generates a health or wellness recommendations for the user by using at least one of a programmed model, decision tree, or one or more machine learning models. The health or wellness recommendation may include at least one of a nutrient recommendation, meal composition recommendation, meal timing commendation, physical activity recommendation, mental activity recommendation, sleep recommendation, health supplement recommendation, health article recommendation, or daily task recommendation.

Other aspects of the present disclosure related to systems for calculating a health value for an individual. In accordance with one implementation, the system includes at least one processor configured to provide at least one graphical user interface to a user that is configured to receive user inputs for one or more of a device selection, a device prioritization, and a data prioritization. The at least one processor may also be configured to create, using application program interfaces (APIs), persistent connections to collect health data from one or more devices, wherein a first prioritization for the connected devices is stored based on user input received through the at least one graphical user interface, the first prioritization for the connected devices defining an order for collecting health data from the connected devices. The at least one processor may also be configured to store, based on further user input received through the at least one graphical user interface, a first prioritization for the data, the first prioritization for the data defining an order among the data from the connected devices for selecting and applying one or more health data parameters. Additionally, the at least one processor may be configured to receive, using the persistent connections, health data from the devices in accordance with the first prioritization for the devices, and calculate a health value for the user using one or more health data parameters based on the received health data, wherein the health data parameters are selected and applied according to the first prioritization for the data. By way of example, the health value may be a biological age or metabolic scale of the user.

Implementations related to above system may include one or more of the following features. For example, using the persistent connections, the health data may be collected periodically from the one or more connected devices in the order defined by the first prioritization for the connected devices. Additionally, or alternatively, the health data may be collected in response to a triggering event. The triggering event may cause the health data to be automatically collected from the one or more connected devices in the order defined by the first prioritization for the connected devices. Additionally, or alternatively, the triggering event may cause the health data to be collected by prompting the user to manually enter health data through the at least one graphical user interface. The triggering event may be defined based on at least one of a condition, event, or schedule. By way of example, the triggering event may be defined based on at least one of: a period of time since the last collection of health data, a request for a health value, a request for a health or wellness recommendation, an authentication of the user, a connection or reconnection of a device, a data synchronization event or schedule, or an expiry of one or more health data parameters.

According to still further aspects of the system, the user input received through the at least one graphical user interface may include manually entered health data for a user. The manually entered health data may include one or more health parameters. Through the at least one graphical user interface, the user may also prioritize manually entered health data or one or more health data parameters. Additionally, the first prioritization for the data may define an order for selecting and applying health data parameters among the health data received from the connected devices and manually entered health data received from the user through the at least one graphical user interface.

The health data may include user data and health data parameters including one or more of age, gender, ethnicity, location, quality of life measures, dietary intake and preferences, weight, height, arm circumference, waist circumference, systolic blood pressure, diastolic blood pressure, total blood pressure, high-density lipoprotein (HDL) cholesterol, low-density lipoprotein (LDL) cholesterol, total cholesterol, oxidized LDL (oxLDL) levels, triglyceride levels, plasma glucose levels, plasma insulin or c-peptide levels, non-esterified fatty acids (NEFA) levels, blood pressure, activity level, moderate muscle strength, vigorous muscle strength, walking distance per time unit, body fat percentage, squat strength, pushup strength, knee pushup strength, plank strength, balance tests, Short Physical Performance Battery (SPPB) score, sleep patterns, blood oxygen saturation (SpO2) level, maximum rate of oxygen (VO2), physical activity patterns, calorie consumption, resting heart rate, breathing rate, respiratory rate, health diseases, family history of health diseases, pregnancy, breast feeding patterns, pregnancy complications, contraceptive practices, perceived stress level, immune markers, vitamin and mineral levels, food intolerance, allergies or allergen markers, microbiome composition and markers, and genotypic or epigenetic information.

According to further aspects of the system, the first prioritization for the connected devices is defined based on an order in which connections to the devices are established. The at least one processor of the system may be further configured to store a second prioritization for the connected devices in response to further user input received through the at least one graphical user interface, the second prioritization for the connected devices being different from the first prioritization for the connected devices that defines an order for collecting health data from the connected devices.

The at least one processor of the system may be further configured to store a second prioritization for the data in response to further user input received through the at least one graphical user interface, the second prioritization for the data being different from the first prioritization for the data that defines an order among the data from the connected devices or manually entered data for selecting and applying one or more health data parameters.

In still further implementations, the at least one processor of the system may be further configured to parse the received health data to obtain the one or more health data parameters. The parsing of the received health data may include applying one or more extract, transform, load (ETL) operations to the received health data to obtain the one or more health data parameters. Additionally, the at least one processor may be configured to apply the one or more health data parameters to generate a health or wellness recommendation for the user. The one or more health data parameters may be applied to or by a health engine that generates a health or wellness recommendations for the user by using at least one of a programmed model, decision tree, or one or more machine learning models. The health or wellness recommendation may include at least one of a nutrient recommendation, meal composition recommendation, meal timing commendation, physical activity recommendation, mental activity recommendation, sleep recommendation, health supplement recommendation, health article recommendation, or daily task recommendation.

Other aspects of the present disclosure relate to a computer readable medium comprising one or more instructions executable by at least one hardware processor. In some implementations, the instructions cause the at least one hardware processor to provide at least one graphical user interface to a user that is configured to receive user input for one or more of a device selection, a device prioritization, and a data prioritization, and create, using application program interfaces (APIs), persistent connections to collect health data from one or more devices, wherein a first prioritization for the connected devices is stored based on user input received through the at least one graphical user interface, the first prioritization for the connected devices defining an order for collecting health data from the connected devices.

The instructions of the computer readable medium may further cause the at least one hardware processor to store, based on further user input received through the at least one graphical user interface, a first prioritization for the data, the first prioritization for the data defining an order among the data from the connected devices for selecting and applying one or more health data parameters. Additionally, the instructions of the computer readable medium may further cause the at least one hardware processor receive, using the persistent connections, health data from the devices in accordance with the first prioritization for the devices and calculate a health value for the user using one more health data parameters based on the received health data, wherein the health data parameters are selected and applied according to the first prioritization for the data. By way of example, the calculated health value may be a biological value or metabolic scale of the user.

In accordance with still further embodiments, a system of one or more computers or processors can be configured to perform particular operations or actions by virtue of having software, firmware, hardware, or a combination of them installed on the system that in operation causes or cause the system to perform the actions. One general aspect includes a computer-implemented system for collecting and prioritizing health data of a user and calculating a biological age and/or other health value(s) for the user based on one or more health data parameters from the collected and prioritized health data, consistent with embodiments of the present disclosure.

A still further general aspect includes a computer-implemented system for providing health or wellness recommendations for a user based on received health data, the received health data including one or more health data parameters of the user. The one or more health data parameters may be applied to or by a health engine that generates the health or wellness recommendations for the user by using at least one of a programmed model, decision tree, or one or more machine learning models. Other embodiments of this aspect include corresponding computer-implemented methods executed by computer systems, apparatus, and computer programs recorded on one or more computer storage devices.

Embodiments of the present disclosure also include computer-implemented methods and computer-readable media with instructions executed by at least one processor to provide steps and features corresponding to the above-mentioned systems and the elements, operations, and aspects thereof.

The above summary and following detailed description are provided for purposes of illustration and do not limit the present disclosure, example embodiments, or claims presented herein.

### BRIEF DESCRIPTION OF THE DRAWINGS

The following drawings which comprise a part of this specification, illustrate several embodiments of the present disclosure and, together with the description, serve to explain the principles and features of the disclosed embodiments. In the drawings:

FIG. 1 is a schematic representation of an example computer-implemented system for health data collection and management, consistent with embodiments of the present disclosure.

FIG. 2 illustrates an example computing device which may be employed in connection with the example system of FIG. 1 and other embodiments of the present disclosure.

FIG. 3 illustrates an example computer-implemented system for health data collection and management, consistent with embodiments of the present disclosure.

FIG. 4 illustrates a flowchart of an example method for persistent health data collection and multi-level prioritization, consistent with embodiments of the present disclosure.

FIG. 5 illustrates an example data ingest and processing method, consistent with embodiments of the present disclosure.

FIG. 6 illustrates an example graphical user interface for connecting and prioritizing devices, consistent with embodiments of the present disclosure.

FIG. 7 illustrates an example graphical user interface for user sign in and authentication, consistent with embodiments of the present disclosure.

FIG. 8 illustrates an example graphical user interface for viewing and updating a prioritization for connected devices, consistent with embodiments of the present disclosure.

FIG. 9 illustrates an example graphical user interface for synchronizing and managing data, consistent with embodiments of the present disclosure.

FIG. 10 illustrates an example graphical user interface for viewing health data parameters, consistent with embodiments of the present disclosure.

FIG. 11 illustrates an example graphical user interface for entering one or more health data parameters and managing a prioritization for data, consistent with embodiments of the present disclosure.

FIGS. 12A and 12B illustrates examples of graphical user interfaces for providing a user with a personalized health value and notifications including personalized health or wellness recommendations, consistent with embodiments of the present disclosure.

FIG. 13 illustrates a flowchart of an example method for calculating a biological age, consistent with embodiments of the present disclosure.

FIG. 14 illustrates a flowchart of an example method for providing a personalized recommendation, consistent with embodiments of the present disclosure.

### DETAILED DESCRIPTION

Example embodiments are described below with reference to the accompanying drawings. The figures are not necessarily drawn to scale. While examples and features of disclosed principles are described herein, modifications, adaptations, and other implementations are possible without departing from the spirit and scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items or meant to be limited to only the listed item or items. It should also be noted that as used herein and in the appended claims, the singular forms “a,” “an,” and “the” include plural references unless the context clearly dictates otherwise.

In the following description, various working examples are provided for illustrative purposes. However, it will be appreciated that the present disclosure may be practiced without one or more of these details.

Throughout this disclosure there are references to “disclosed embodiments,” which refer to examples of inventive ideas, concepts, and/or manifestations described herein. Many related and unrelated embodiments are described throughout this disclosure. The fact that some “disclosed embodiments” are described as exhibiting a feature or characteristic does not mean that other disclosed embodiments necessarily share that feature or characteristic.

Embodiments described herein include non-transitory computer readable medium containing instructions that when executed by at least one processor, cause the at least one processor to perform a method or set of operations. Non-transitory computer readable mediums may be any medium capable of storing data in any memory in a way that may be read by any computing device with a processor to carry out methods or any other instructions stored in the memory. The non-transitory computer readable medium may be implemented as software, firmware, hardware, or any combination thereof. Software may preferably be implemented as an application program tangibly embodied on a program storage unit or computer readable medium consisting of parts, or of certain devices and/or a combination of devices. The application program may be uploaded to, and executed by, a machine comprising any suitable architecture. Preferably, the machine may be implemented on a computer platform having hardware such as one or more central processing units (“CPUs”), a memory, and input/output interfaces. The computer platform may also include an operating system and microinstruction code. The various processes and functions described in this disclosure may be either part of the microinstruction code or part of the application program, or any combination thereof, which may be executed by a CPU, whether or not such a computer or processor is explicitly shown. In addition, various other peripheral units may be connected to the computer platform such as an additional data storage unit and a printing unit. Furthermore, a non-transitory computer readable medium may be any computer readable medium except for a transitory propagating signal.

The memory may include any mechanism for storing electronic data or instructions, including Random Access Memory (RAM), a Read-Only Memory (ROM), a hard disk, an optical disk, a magnetic medium, a flash memory, other permanent, fixed, volatile or non-volatile memory. The memory may include one or more separate storage devices collocated or disbursed, capable of storing data structures, instructions, or any other data. The memory may further include a memory portion containing instructions for the processor to execute. The memory may also be used as a working memory device for the processors or as a temporary storage.

Some embodiments may involve at least one processor. A processor may be any physical device or group of devices having electric circuitry that performs a logic operation on input or inputs. For example, the at least one processor may include one or more integrated circuits (IC), including application-specific integrated circuit (ASIC), microchips, microcontrollers, microprocessors, all or part of a central processing unit (CPU), graphics processing unit (GPU), digital signal processor (DSP), field-programmable gate array (FPGA), server, virtual server, or other circuits suitable for executing instructions or performing logic operations. The instructions executed by at least one processor may, for example, be pre-loaded into a memory integrated with or embedded into the controller or may be stored in a separate memory.

In some embodiments, the at least one processor may include more than one processor. Each processor may have a similar construction, or the processors may be of differing constructions that are electrically connected or disconnected from each other. For example, the processors may be separate circuits or integrated in a single circuit. When more than one processor is used, the processors may be configured to operate independently or collaboratively. The processors may be coupled electrically, magnetically, optically, acoustically, mechanically or by other means that permit them to interact.

Embodiments consistent with the present disclosure may involve a network. A network may constitute any type of physical wired or wireless networking arrangement used to exchange data, commands, signaling information, and so on. For example, a network may be the Internet, a private data network, a virtual private network using a public network, a Wi-Fi network, a local area network (“LAN”), a wide area network (“WAN”), and/or other suitable connections that may enable information exchange among various components of the system. In some embodiments, a network may include one or more physical links used to exchange data, such as Ethernet, coaxial cables, twisted pair cables, fiber optics, or any other suitable physical medium for exchanging data. A network may also include one or more networks, such as a private network, a public switched telephone network (“PSTN”), the Internet, and/or a wireless cellular or mobile network (e.g., LTE, 4G, 5G). A network may be a secured network or unsecured network. In other embodiments, one or more components of the system may communicate directly through a dedicated communication network. Direct communications may use any suitable technologies, including, for example, BLUETOOTH™, BLUETOOTH LE™ (BLE), Wi-Fi, near field communications (NFC), or other suitable communication methods that provide a medium for exchanging data and/or information between separate entities.

In some embodiments, machine learning networks or algorithms may be trained using training examples, for example in the cases described below. Some non-limiting examples of such machine learning algorithms may include predictive models, support vector machines, random forests, nearest neighbor algorithms, deep learning algorithms, artificial neural network algorithms, convolutional neural network algorithms, recursive neural network algorithms, linear machine learning models, non-linear machine learning models, ensemble algorithms, and so forth. For example, a trained machine learning network or algorithm may comprise an inference model, such as a predictive model, a classification model, a regression model, a clustering model, a segmentation model, an artificial neural network (such as a deep neural network, a convolutional neural network, a recursive neural network, etc.), a random forest, a support vector machine, and so forth. In some examples, the training examples may include example inputs together with the desired outputs corresponding to the example inputs. Further, in some examples, training machine learning algorithms using the training examples may generate a trained machine learning algorithm, and the trained machine learning algorithm may be used to estimate outputs for inputs not included in the training examples. The training may be supervised or non-supervised, or a combination thereof. In some examples, engineers, scientists, processes and machines that train machine learning algorithms may further use validation examples and/or test examples. For example, validation examples and/or test examples may include example inputs together with the desired outputs corresponding to the example inputs, a trained machine learning algorithm and/or an intermediately trained machine learning algorithm may be used to estimate outputs for the example inputs of the validation examples and/or test examples, the estimated outputs may be compared to the corresponding desired outputs, and the trained machine learning algorithm and/or the intermediately trained machine learning algorithm may be evaluated based on a result of the comparison. In some examples, a machine learning algorithm may have parameters and hyper-parameters, where the hyper-parameters are set manually by a person or automatically by a process external to the machine learning algorithm (such as a hyper parameter search algorithm), and the parameters of the machine learning algorithm are set by the machine learning algorithm according to the training examples. In some implementations, the hyper-parameters are set according to the training examples and the validation examples, and the parameters are set according to the training examples and the selected hyper-parameters. The machine learning networks or algorithms may be further retrained based on any output.

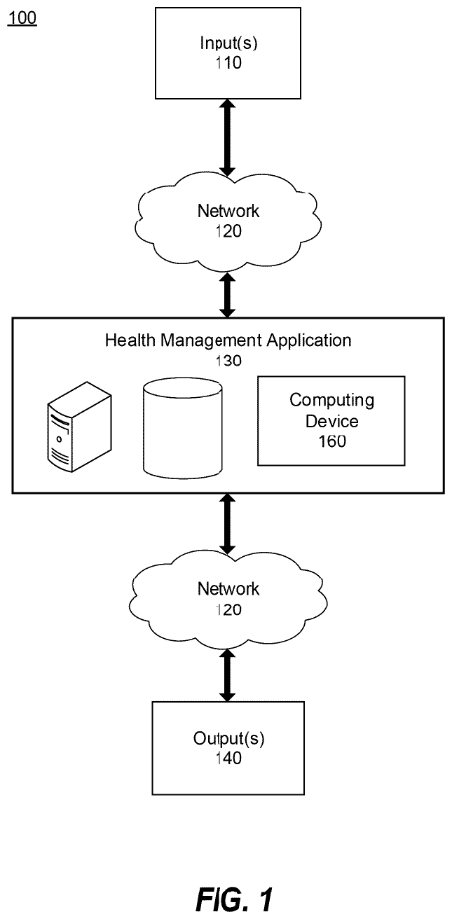

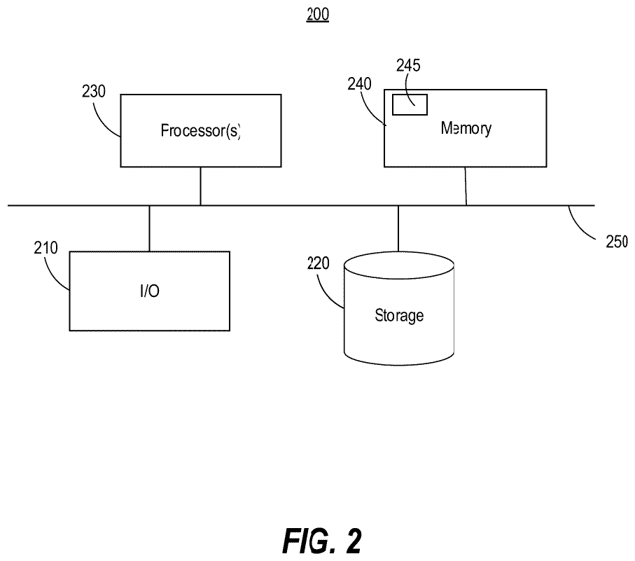

FIG. 1 illustrates an example computer-implemented system 100 for health data collection and management, according to embodiments of the present disclosure. As shown in FIG. 1, system 100 may comprise a health management application 130. “Application” as used herein may refer broadly to any set of electronic instruction, whether commonly known as software, firmware, middleware, microcode, hardware description language, or otherwise. Examples of an application include serverless code instances, scripts, and programs. Applications may comprise one or more source code instructions written in a software application language that may be translated into executable, binary, or any other machine-readable code. Applications may be executable by one or more hardware processors. Examples of software application languages include Python, JavaScript, Java, C, C++, C#, Ruby, although a software application may be written in any other language or format. Health management application 130 may be executed by and/or in electronic communication with one or more servers, databases, and computing devices (such as computing device 160 or computing device 200 described in more detail in connection with FIG. 2), which may be used to perform the functions and operations described in more detail herein. In some implementations, health management application 130 includes applications executed by one or more servers (e.g., networked or cloud-based servers) and applications executed on computing devices of users (e.g., mobile phones, computers, laptops, tablets, smartwatches, and so on).

As shown in FIG. 1, health management application 130 may be in electronic communication with one or more network(s) 120. Network(s) 120 may be or include any electronic communication channel, such as the Internet, a local area network, a wide area network, a mobile network (e.g., LTE, 4G, 5G), Wi-Fi, or BLUETOOTH™, as explained above. Network(s) 120 may also include collections or combinations of one or more networks, such as those listed above and implemented through wired and/or wireless networks. Through network(s) 120, health management application 130 may receive one or more inputs 130, and it may transmit one or more outputs 140.

Input(s) 110 may comprise data or information that may be used by health management application 130 to perform the functions and operations described herein. Non-limiting examples of inputs that may be received by health management application 130 include user data, non-user data, user health data, client device data, database data, metadata, API requests, API responses, or any other data. In some implementations, data may include combinations of different data types (e.g., received health data may include user data and one or more health data parameters). Input(s) 110 may be entered manually by a user or automatically under the control of software or programmed processes, for example. Input(s) 110 may include original data (e.g., data entered upon the registration of a user), corrected data (e.g., to revise earlier entered data), and/or updated data (e.g., after gathering more data or revised data based on intervention or following health recommendations). As disclosed herein, one or more graphical user interfaces (GUIs) may be provided to enable the collection of input(s) 110, including data manually entered or selected by a user through GUI(s) presented on their computing device (e.g., a mobile phone or tablet). Further, input(s) 110 may be entered separately, collectively, and/or periodically. In some implementations, input(s) 110 may be provided from one application to another application (e.g., one health application (such as Apple Health or HealthKit) to health management application 130), and/or from one device to another device (such as a user's computing device to a server executing the health management application 130).

Output(s) 140 may comprise data or information, whether intermediary or final, resulting from the functions and operations described herein. Non-limiting examples of outputs that may be generated by health management application 130 include calculated health values (such as a user's biological age or metabolic scale) and health or wellness recommendations such as personalized diet patterns, personalized food recommendations (“hero foods”), meal timings, recommended supplements, sleep pattern suggestions, articles, and stress management recommendations, among others. Output(s) 140 may be provided in response to processing data and/or performing functions or processes, consistent with the present disclosure. Output(s) 140 may include original output(s) (e.g., an original set of output(s) generated in response to input(s) of data first entered by a user), corrected output(s) (e.g., output(s) generated in response to revised input(s) for earlier entered data), and/or updated output(s) (e.g., output(s) generated in response to input(s) of more data or revised data following one or more health and/or wellness recommendations followed by a user). Output(s) 140 may be electronically displayed or sent to users. In some embodiments, output(s) 140 are presented to a user through one or more GUIs or graphical displays on a user's computing device (e.g., mobile phone, computer, laptop, tablet, smartwatch, and so on). Further, output(s) 140 may be provided separately, collectively, and/or periodically. As with input(s) 110, output(s) 140 may be provided from one application to another application and/or from one device to another device.

FIG. 2 illustrates an example computing device 200 for use with health management application 130 of FIG. 1, consistent with embodiments of the present disclosure. Computing device 200 may be used in connection with the implementation of the example system of FIG. 1 (including, e.g., computing device 160) as well as the example methods and operations described herein. It is to be understood that in some embodiments the computing device may include multiple sub-systems, such as cloud computing systems, servers, and/or any other suitable components for receiving and processing data. Also, the number and arrangement of components shown in FIG. 2 are exemplary. It will be appreciated that modifications, additions, and substitutions can be made without departing from the principles and aspects of the present disclosure.

As shown in FIG. 2, computing device 200 may include one or more processor(s) 230, which may include, for example, one or more integrated circuits (IC), including application-specific integrated circuit (ASIC), microchips, microcontrollers, microprocessors, all or part of a central processing unit (CPU), graphics processing unit (GPU), digital signal processor (DSP), field-programmable gate array (FPGA), server, virtual server, or other circuits suitable for executing instructions or performing logic operations, as noted above. In some embodiments, processor(s) 230 may include, or may be a component of, a larger processing unit implemented with one or more processors. The one or more processors 230 may be implemented with any combination of general-purpose microprocessors, microcontrollers, digital signal processors (DSPs), field programmable gate array (FPGAs), programmable logic devices (PLDs), controllers, state machines, gated logic, discrete hardware components, dedicated hardware finite state machines, or any other suitable entities that can perform calculations or other manipulations of information. In some embodiments, health management application 130 of FIG. 1 is executed in whole or part by computing device 200 with one or more processors 230.

As further shown in FIG. 2, processor(s) 230 may be communicatively connected via at least one bus or network 250 to a memory 240. Bus or network 250 may be adapted to communicate data and other forms of information. Memory 240 may include a memory portion 245 that contains instructions that when executed by the processor(s) 230, perform the operations and methods described in detail herein. Memory 240 may also be used as a working memory for processor(s) 230, a temporary storage, and other memory or storage roles, as the case may be. By way example, memory 240 may be a volatile memory such as, but not limited to, random access memory (RAM), or non-volatile memory (NVM), such as, but not limited to, flash memory. As will be appreciated, more than one memory 240 may be provided as part of the computing device 200.

Processor(s) 230 may also be communicatively connected via bus or network 250 to at least one I/O device 210. I/O device 210 may include any type of input and/or output device or periphery device. I/O device 210 may include one or more network interface cards, APIs, data ports, and/or other components for supporting connectivity with processor(s) 230 via network 250.

As further shown in FIG. 2, processor(s) 230 and the other components (210, 240) of computing device 200 may be communicatively connected to at least one database or storage device 220. Storage device 220 may electronically store data in an organized format, structure, or set of files. Storage device 220 may include a database management system to facilitate data storage and retrieval. While illustrated in FIG. 2 as a single device, it is to be understood that storage device 220 may include multiple devices either collocated or distributed. In some embodiments, storage device 220 may be implemented on a remote network, such as a cloud storage.

Processor(s) 230 and/or memory 240 may also include machine-readable media for storing software or sets of instructions. “Software” as used herein refers broadly to any type of instructions, whether referred to as software, firmware, middleware, microcode, hardware description language, or otherwise. Instructions may include code (e.g., in source code format, binary code format, executable code format, or any other suitable format of code). The instructions, when executed by one or more processors 230, may cause the processor(s) to perform the various operations and functions described in further detail herein.

Implementations of computing device 200 are not limited to the example embodiment shown in FIG. 2. The number and arrangement of components (e.g., 210, 220, 230, 240) in FIG. 2 may be modified and rearranged. Further, while not shown in FIG. 2, computing device 200 may be in electronic communication with other network(s), including the Internet, a local area network, a wide area network, a metro area network, and other networks capable of enabling communication between the elements of the computing architecture. Also, computing device 200 may retrieve data or other information described herein from any source, including storage device 220 as well as from network(s) or database(s). Further, computing device 200 may include one or more machine-learning models used to implement the neural networks described herein and may retrieve or receive weights or parameters of machine-learning models, training information or training feedback, and/or any other data and information described herein.

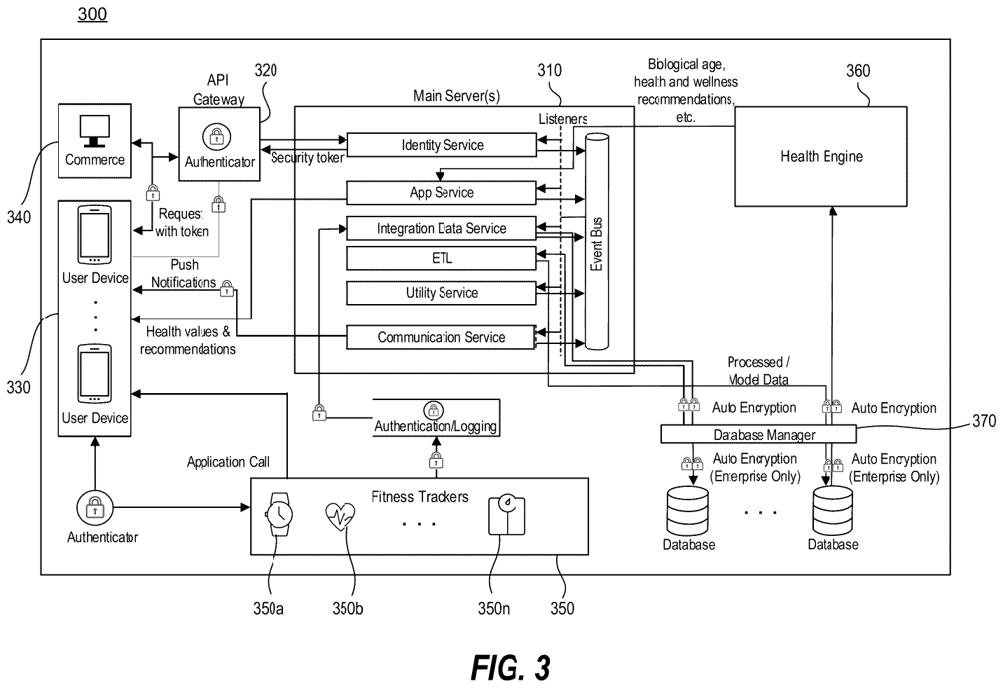

FIG. 3 illustrates an example computer-implemented system 300 for collecting, managing, and processing health data of users and providing health values and/or other output(s) such as health or wellness recommendations, consistent with embodiments of the present disclosure. Similar to the example system 100 of FIG. 1, the example system 300 of FIG. 3 may be implemented with one or more processors (e.g., processor(s) 230 of the computing device of FIG. 2) and/or non-transitory computer readable medium, such as a CPU, FPGA, ASIC, or any other processing structure(s) or storage medium. In some embodiments, system 300 may be implemented as a centralized or cloud-based service over the Internet. In further embodiments, system 300 may be implemented as a distributed system with one or more processor(s) or server(s) that are networked to facilitate health data collection, management, and processing for a plurality of users. Although not shown in FIG. 3, system 300 may also include one or more applications (“apps”) that are executable on mobile phones, laptops, tablets, smartwatches and/or other computing devices of users (user devices 330) to enable data collection, management, and processing of input(s) and output(s) of the system. To support system 300 and the services to users, one or more GUIs and/or graphical displays may be implemented and presented to users on their user devices 330, as further disclosed herein.

As shown in the example embodiment of FIG. 3, system 300 may include a main server 310, an application programming interface (API) gateway 320, one or more user devices 330, an e-commerce platform 340, one or more fitness trackers 350 and/or other devices or sources providing health data, a health engine 360, and a database manager 370. User devices 330 may include one or more computing devices (see, e.g., FIG. 2), such as a mobile phone, a tablet, a personal computer, a laptop, a smartwatch, and so on. Fitness trackers 350 may include a variety of health and fitness trackers that are worn or used by users, including fitness tracking bands, rings, watches, monitors, health apps, and other devices. Several non-limiting examples are illustrated in FIG. 3. As further disclosed herein, through a health management application executed on each user device 330, a user may select one or more fitness trackers 350 and/or other devices to connect to and share their health data. One or more graphical user interfaces and/or other I/O interfaces (e.g., a display, keyboard, mouse, etc) may be used by a user to provide user inputs to prioritize the devices and prioritize the specific data (based on device or other source) that should be selected and applied to calculate health values and provide health or wellness recommendations by system 300. In some implementations, health values (such as a user's biological age or metabolic scale) are computed by health engine 360. Health engine 360 may also apply a user's health data to generate health or wellness recommendations (such as diet or exercise recommendations) using one or more programmed models, decision trees, and/or machine learning models. It is to be understood that the elements shown in FIG. 3 are exemplary only and are not intended to be exhaustive of the example system 300. Additional or fewer components than those shown in FIG. 3 may be used, depending on the specific application, purpose, or implementation.

Main server 310 may comprise one or more computing devices, such as one or more servers or processors, and may be configured to perform operations through executable applications or software (e.g., machine-readable instructions). Main server 310 may maintain states, handle events, and initiate actions of the example system 300. As shown in FIG. 3, main server 310 may comprise an identity service layer for authenticating users and managing access to resources and service; an application service layer for managing the operations of health engine 360 for user devices 330; an integration data service layer for incorporating data from various sources; an ETL layer for extracting, transforming, and loading health data from fitness trackers 350 and/or other devices or sources of data; a utility service layer for managing non-business-related logic; and a communication service layer for managing data transfers between health engine 360 and other components or devices of system 300 (e.g., user devices 330 or database manager 370). Additional or fewer components than those shown in FIG. 3 may be part of the main server 310.

Main server 310 may interface with API gateway 320 for receiving requests and transmitting responses of API calls. API gateway 320 may comprise a plurality of application program interfaces (APIs) to facilitate the collection and communication of data and messages between system components, devices, and applications. Although not shown in FIG. 3, each of the user devices 330 may also include one or more APIs to support connectivity by and between components, devices and applications including those that are resident to, executed by, or in communication with each user device (such as fitness trackers 350). The arrangement of APIs may depend on system requirements and other factors, such as the location and type of connected devices. For example, if the connected device is hosted on a network or cloud location, the API gateway 320 may be utilized to facilitate the connection and collection of data from that resource. On the other hand, of the connected device is local to or hosted on one of the user devices 330 (such as a third-party health app), it may be beneficial to have APIs on that user device to support and enable the connection and collection of data. As will be appreciated, any suitable combination of APIs may be utilized. Referring again to FIG. 3, API gateway 320 may perform functions to ensure proper handling of requests and responses, such as authentication of user devices 330 issuing API requests or to validate actions with e-commerce platform 340 (e.g., purchase of health equipment, supplements and other items). Main server 310 may also communicate data and information with user devices 330, including input(s) and output(s), using one or more applications, GUIs and/or graphical displays executed on each user device 330. For example, as shown in FIG. 3, main server 310 may transmit “Push Notifications” via the communication service layer to alert a user of user device 330 of certain information (e.g., an update, alert, or message). Additionally, main server 310 may receive from a user device a “Request with Token” for purposes of exchanging security tokens and authenticating the user of user device 330. In some implementations, secure connections and data encryption are used to maintain security and privacy. For example, a variety of security measures may be employed, such as unique user identifications (UIID) using a 128-bit value, application logins based on multi-factor authentication (MFA), and/or encryption and decryption using algorithms such as Secure Hash Algorithm (SHA) or Advanced Encryption Standard (AES) 256-bit to securely protect data. Further, the application service layer of main server 310 may provide health values and/or health or wellness recommendations from health engine 360 to the user of user device 330 to aid that user in health or wellness improvement.

As disclosed herein, main server 310 may obtain data from or through user devices 330, such as user inputs and/or health data, to perform the operations described herein. Main server 310 or user devices 330 may also create persistent connections with one or more fitness trackers 350 and/or other devices to obtain input(s) in the form of user health data (e.g., weight, heart rate, blood pressure, blood oxygen saturation level, etc, and activity information, such as cardiac information, sleep duration and quality levels, number of steps walked during a session (e.g., the past day, past week, or past month), and any other health or wellness information) captured or computed by fitness trackers 350 and/or other connected devices or sources of data. Connections are made “persistent” by virtue of authenticating user's access to a device and maintaining that connection (i.e., “keep alive”) as long as the device and attendant network channel(s) are available. As disclosed herein, APIs may be used to facilitate the communication of data over such connections. In the event that the connection is interrupted or temporarily lost, main server 310 will automatically attempt to reestablish the connection with the device. If the connection is not reestablished within a predetermined number of attempts, main server 310 will again attempt to make the connection when a data collection or synchronization is next made or triggered. Notifications may be provided or displayed to a user (e.g., via a GUI or graphical display on their user device) to alert them of a lost connection with a particular device. In the event that the connection with a device cannot be reestablished, main server 310 will collect data from the next device according to the prioritization information (device and/or data level) for the user.

As used herein, the term “health data” refers to all forms of health or wellness data associated with a user. Health data may also include user data and activity data. By way of non-limiting example, health data may include one or more of: age, gender, ethnicity, location, quality of life measures, dietary intake and preferences, weight, height, arm circumference, waist circumference, systolic blood pressure, diastolic blood pressure, total blood pressure, high-density lipoprotein (HDL) cholesterol, low-density lipoprotein (LDL) cholesterol, total cholesterol, oxidized LDL (oxLDL) levels, triglyceride levels, plasma glucose levels, plasma insulin or c-peptide levels, non-esterified fatty acids (NEFA) levels, blood pressure, activity level, moderate muscle strength, vigorous muscle strength, walking distance per time unit, body fat percentage, squat strength, pushup strength, knee pushup strength, plank strength, balance tests, Short Physical Performance Battery (SPPB) score, sleep patterns, blood oxygen saturation (SpO2) level, maximum rate of oxygen (VO2), physical activity patterns, calorie consumption, resting heart rate, breathing rate, respiratory rate, health diseases, family history of health diseases, pregnancy, breast feeding patterns, pregnancy complications, contraceptive practices, perceived stress level, immune markers, vitamin and mineral levels, food intolerance, allergies or allergen markers, microbiome composition and markers, and genotypic or epigenetic information. Consistent with the present disclosure, “health data” may include any combination of attributes from one or more categories, including metabolic, cardiovascular, muscle, and/or immunity attributes. Further, health data may be collected from numerous sources and in numerous ways. For example, health data may be collected from one or more connected devices or manually entered by a user, as disclosed herein, In some embodiments, health data may also be collected from one or more affirmative or negative responses by a user to one or more health-related questions (e.g., “what is your age” or “what is your weight” or “are you a smoker?” or “are you pregnant?”). In this manner, health data may be collected from each individual through a questionnaire or other list of questions displayed via a health management software application (“app”) or program running on, e.g., a user's mobile phone, laptop, tablet, computer, etc. Health data may also be received through online questionnaires managed through one or more websites or through studies or surveys organized by a clinic, health care provider, or organization. Health data may also be electronically stored and received from a database or memory device. In some embodiments, health data are stored as part of enrollment data, survey data, population data, and/or personal health records. Health data may also be corrected, revised, or estimated (e.g., in case of incomplete or incorrect entry of such data) and may also be updated (e.g., in case of changes to one or more user health data due to monitoring (such as by a fitness tracker or other device) or intervention and/or the following of health recommendations by a user and the improvement to their health data). The above examples are provided for illustration purposes only and are not intended to be exhaustive.

As used herein, the term “devices” encompasses all forms of devices (including fitness trackers 350) and other sources that provide health data and/or other user data (such as applications, health records, test data or results, lab reports, and databases). Accordingly, “devices” that provide health data are not limited to hardware or electronic devices. In the example embodiment of FIG. 3, fitness trackers 350 are shown as including one or more types of devices such as a wearable device 350a, a heart rate monitor 350b, . . . and/or a weight scale 350n. These are non-limiting examples. Other examples of fitness trackers 350 or devices that provide health data include health apps (e.g., Apple Health or HealthKit), fitness bands and smartwatches (e.g., Fitbit, Garmin, and so on), keto and blood glucose monitors (e.g., Keto-Mojo meter, ketone urinalysis devices, and ketone breath devices), health monitoring rings, blood-based devices, and so on. For some devices, a user's login and/or authentication credentials may be required to enter and/or access health data and/or other user data. After such information is successfully entered and exchanged (including keys or tokens), it is used to main the persistent connection with the device. Other privacy controls may also be implemented, such as multi-factor authentication (at the time of first log-in and thereafter periodically or conditioned on an event, such as a reconnection) and secure storage.

Consistent with embodiments of the present disclosure, multi-level prioritization may be provided to prioritize devices for data collection and to prioritize the specific health data or parameters for generating health values and other related information, such as health or wellness recommendations. The prioritization may be configured by each user and unique to each user and their preferences. For example, as disclosed herein, each user may prioritize the connected devices for collecting data and prioritize the data to be selected and applied for generating health values and recommendations. The prioritization for the connected devices may be different from the prioritization for the data (i.e., the manner and order in which data is collected can be different from manner and order in which data is applied). In addition, the prioritization information may be used and applied universally across all determined health values and recommendations. However, in some embodiments, a user may be given the option to select and apply different prioritizations to different health values or recommendations. For example, through user input received via a GUI, a user may designate different prioritizations of data to be used for different calculated health values or generated health or wellness recommendations. In some embodiments, GUIs and/or other graphical displays are provided on each user device 330 to enable the user to provide input(s) for defining each of the above noted prioritizations. The GUIs or other graphical displays may also enable a user to provide inputs to update, change, or modify their preferred prioritizations. User inputs may also include manually entered health data, including one or more health data parameters. Main server 310 may store prioritization information and user data and health-related information through database manager 370. The data of a user may also be securely stored on their user device 330 and periodically synchronized with the data securely stored for each user by database manager 370 (including the one or more databases thereof).

In some embodiments, if a user has not manually entered prioritization information for connected devices or data, then a predetermined prioritization or default prioritization may be applied and stored by main server 310. For example, the prioritization for connected devices may be set such that the first connected device is given the highest priority and descending priority is given to devices connected thereafter. Alternatively, the last connected device may be given the highest priority and descending priority is given to devices connected earlier in time. A similar prioritization default may be applied if a user has not manually entered prioritization information for health data. For example, the prioritization for data may be set as default that the first collected data is given the highest priority and descending priority is given to data collected thereafter. Alternatively, the last collected data may be given the highest priority and descending priority is given to data collected earlier in time. If a user manually enters data, if may treated like any other source of data and prioritization may be assessed based on whether it is the first or last collected data.

Consistent with the present disclosure, a user may set prioritizations for specific health data parameters. These prioritizations may differ from the prioritizations for connected devices. For example, at the connected device level, a user may select to prioritize Apple Health over Fitbit, but at the data level, for one or more specific health data parameters (e.g., resting heart rate), the user may prioritize Fitbit's data over Apple Health's data.

As disclosed herein, the prioritization for connected devices may define an order for collecting health data from the connected devices to a user device. Separately, the prioritization for the data may define an order among the health data from the connected devices and/or manually entered health data for selecting and applying one or more health data parameters. The prioritization for the data may be specifically defined among one or more health data parameters (e.g., weight, heart rate, blood pressure, muscle strength, daily activity, sleep patterns, and so on) according to input from the user and may be different from the prioritization for collecting data from the connected devices. The above multi-level prioritization provides more flexibility and options for connecting devices and how the data is collected from such devices, as well as flexibility and choice among the sources for health data parameters for generating health values and recommendations. Furthermore, a user is also provided with more freedom and control of their personal health data, and the system can provide more accuracy and privacy to users.

Consistent with implementations of the present disclosure, a user's health data may also be managed and kept current more effectively than extant solutions. For example, using the persistent connections, the health data may be collected periodically from the one or more connected devices in the order defined by the prioritization for the connected devices. Additionally, or alternatively, the health data may be collected in response to a triggering event. The triggering event may cause the health data to be automatically collected from the one or more connected devices in the order defined by the prioritization for the connected devices. Still further, the triggering event may cause the health data to be collected by prompting the user to manually enter health data through, for example, a graphical user interface. The triggering event may be defined based on a condition, event, or schedule. For example, the triggering event may be defined based on: a period of time since the last collection of health data, a request for a health value, a request for a health or wellness recommendation, an authentication of the user, a connection or reconnection of a device, a data synchronization event or schedule, and/or an expiry of one or more health data parameters.

As further disclosed herein, the user input received through a graphical user interface may include manually entered health data for a user. The manually entered health data may include one or more health parameters. Through the at least one graphical user interface, the user may also prioritize manually entered health data or one or more health data parameters. As described above, the prioritization for the data may define an order for selecting and applying health data parameters among the health data received from the connected devices and/or manually entered health data received from the user through a graphical user interface. These and other aspects are further disclosed herein.

Referring again to FIG. 3, main server 310 may be in electronic communication with health engine 360. Main server 310 may task health engine 360 to perform operations, such as calculating and providing output(s) in the form of health values (such as biological age or metabolic scale) and health or wellness recommendations. Health engine 360 may include one or more of programmed models, decision trees, and/or machine-learning models (e.g., one or more predictive models) to perform the functions and operations described herein. Example embodiments for implementing health engine 360 and calculating health values and health or wellness recommendations are provided in U.S. Provisional Application No. 63/363,017, filed on Apr. 14, 2022, and titled “Computer-Implemented Systems and Methods for Health Data Analysis and Management,” the entire disclosure of which is hereby incorporated herein.

Main server 310 may also communicate with database manager 370 to store and fetch data in the databases thereof. Database manager 370 may store data associated with system 300, such as various inputs of data (e.g., user-provided information (including user data and health data), device selections and prioritizations, data prioritizations, etc.), outputs of data (e.g., estimated user attributes, biological age and other health values, health or wellness recommendations, etc.), machine learning models or other algorithms, and any other data. In some embodiments, as shown in FIG. 3, database manager 370 (including the one or more databases thereof) may be in communication with health engine 360. For example, database manager 370 may provide data to processing pipelines or trained machine learning models implemented by health engine 360 to calculate health values (such as a user's biological age or metabolic scale), generate health and wellness recommendations, and/or perform other functions and operations of the health engine. Health engine 360 may comprise any suitable combination of hardware, software, and/or firmware, including servers, neural networks, and software-based applications. Health values and recommendations generated by health engine 360 may be stored by or through database manager 370. Main server 310 may relay or coordinate the communication of data and information via the different components of system 300 (e.g., relaying health values and health or wellness recommendations from health engine 360 to database manager 370 and/or user devices 330). In some embodiments, the different components of system 300 may communicate directly with one another.

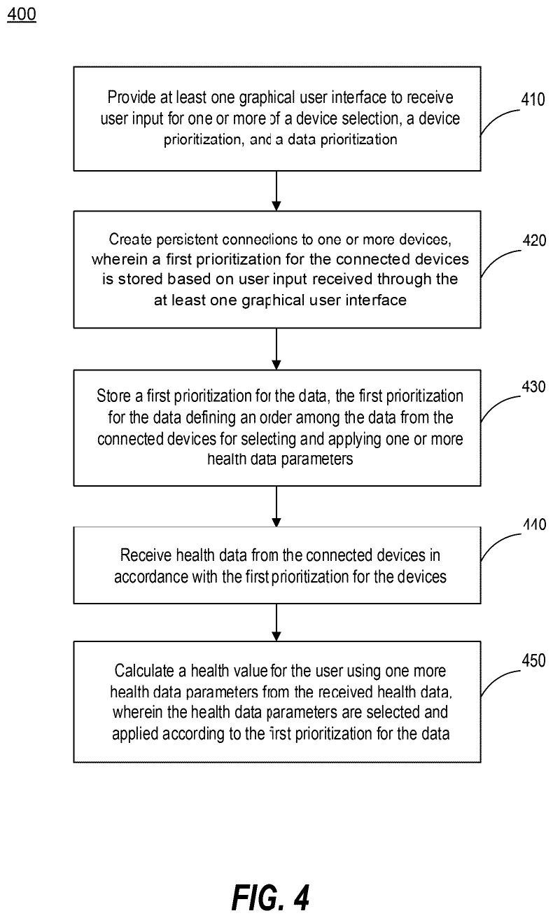

FIG. 4 illustrates a flowchart of an example method 400 for persistent health data collection and multi-level prioritization, consistent with embodiments of the present disclosure. The example method 400 may be implemented using one or more processors (e.g., processor(s) 230 of the computing device of FIG. 2) or non-transitory computer readable medium, such as a CPU, FPGA, ASIC, or any other processing structure(s) or storage medium. In some embodiments, example method 400 may be performed by one or more processor(s) of system 300 of FIG. 3, such as user devices 330, main server 310, health engine 360, and/or database manager 370. It is to be understood that the steps shown in FIG. 4 are exemplary only and are not intended to be exhaustive. The steps shown in FIG. 4 may be modified, rearranged, or combined with other steps, depending on the specific application or purpose.

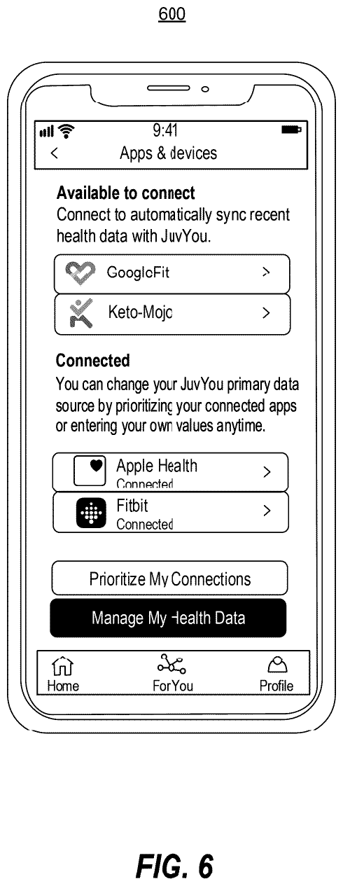

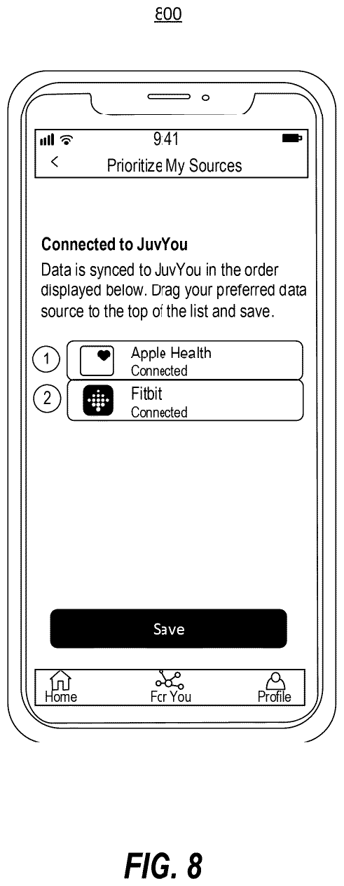

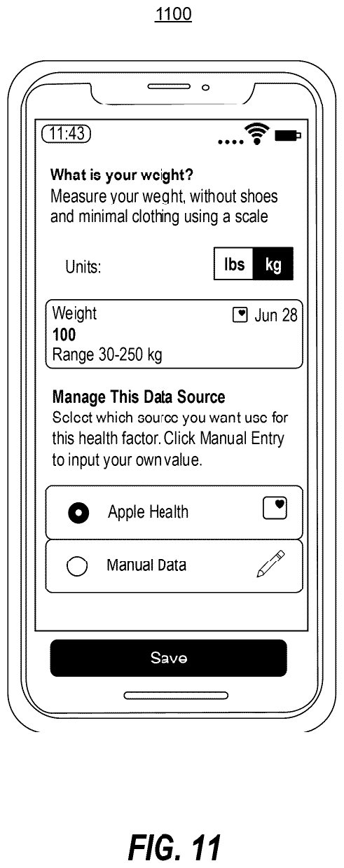

At step 410, at least one processor may provide at least one graphical user interface to receive user input. The user input may include, for example, a device selection, a device prioritization, and/or a data prioritization. By way of example, user input for a device selection may be entered by a user to select a device for connection to the system. To facilitate the user's selection, the at least one graphical user interface or GUI (see, e.g., FIG. 6) may display a list of available devices to connect. The GUI may also provide a list of connected devices so that the user is aware of the devices that are already connected to the system. The devices may provide health data and include, for example, a Fitbit or Garmin device, an Apple or Android smartwatch, a blood glucose monitor, and/or other types of fitness trackers. Devices may also include health applications or data platforms such as Google Fit, Apple Health or Health Kit, Keto-Mojo, and/or other health applications or sources. User input for a device prioritization may be entered by a user to prioritize connected devices for data collection. In some implementations, the prioritization for connected devices may be initially defined based on an order in which the connections to the devices are established. A user may be provided the option to update the prioritization for connected devices (see, e.g., FIG. 6—“Prioritize My Connections”) and make changes to the prioritization order of connection devices through user input such as selections and/or drag and drop operations (see, e.g., FIG. 8—“Drag your preferred data source to the top of the list and save”). In addition, user input for a data prioritization may be entered by a user to prioritize where data (including one or more specific health data parameters) are sourced for calculating health values or providing recommendations. In some implementations, a user may be provided with the option of managing their data (see, e.g., FIG. 6—“Manage My Health Data”) and prioritizing where specific health data parameters (such as weight, heart rate, blood pressure, etc) are sourced for health value calculations and providing recommendations (see, e.g., FIG. 11—“Weight” and “Manage This Data Source”). The user input for data prioritization may include selecting a primary source for one or more health data parameters (e.g., Apple Health) through a graphical user interface. The user input may also include the option to manually enter a health data parameter and select the manually entered value as the primary source over a secondary source for the same health data parameter (e.g., Fitbit, Apple Health or other).

Prioritization information, including the prioritization for devices and data, may be stored locally on the user device 330 of the user and/or stored centrally by main server 310 through database manager 370. As described above, the prioritization information may be unique to each user based on their selections and inputs, and the information may be stored securely. By way of example, the prioritization information may be stored as a set of values or as part of a table. With respect to the prioritization for connected devices, the prioritization information may be used by main server 310 to control the order by which data is collected from the connected devices. If a device is unavailable or data from it is unobtainable, then that device may be skipped and the next device in the priority order may be accessed to collect the user's health data. This process may be followed until all devices have been accessed or checked for data. With respect to the prioritization for data, the prioritization information may be used by main server 310 to control which health data parameters are selected and applied by health engine 360 for calculating a health value or providing a health or wellness recommendation to a user. For example, a user may prioritize a heart rate parameter from his or her connected Fitbit device over any other source for that parameter (e.g., Apple Health, a manually entered value, or another device or source). If the prioritized parameter is unavailable or unobtainable, then main server 310 may use the next prioritized source for that parameter in its place. The prioritization order may be of any length depending on the number of data sources available for a health data parameter. In some implementations, where no prioritization for data is provided by a user, main server 310 may prioritize and select health data parameters based on the prioritization for connected devices or the first available source for each data parameter.

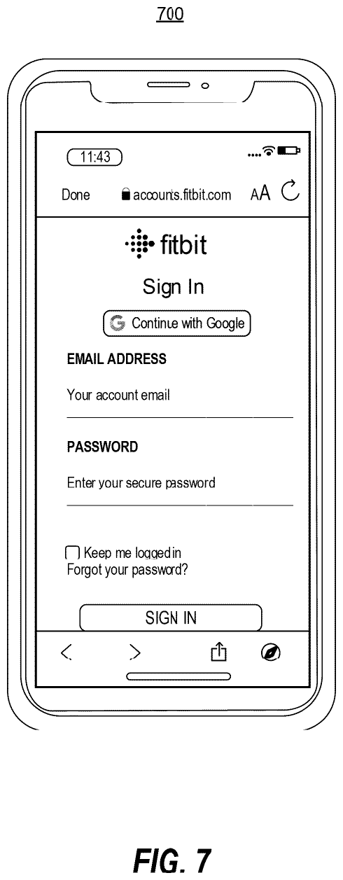

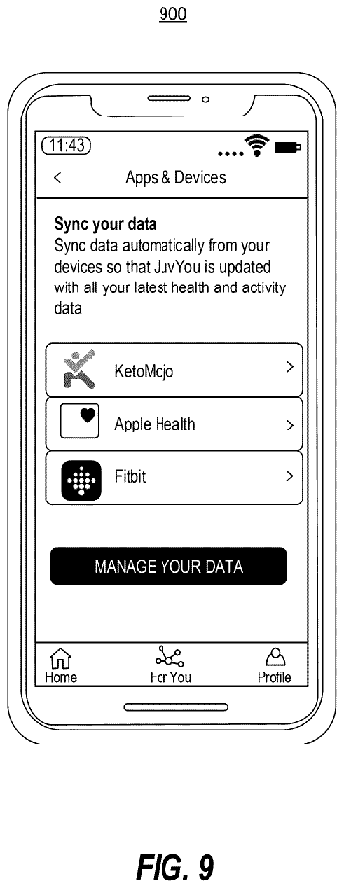

At step 420, the at least one processor may connect to one or more devices to collect health data. The connections may be established automatically and/or in response to user input. As previously explained, a user may select which devices are to be connected to the system. Main server 310 may then establish the connection (e.g., using one or more authenticators and/or APIs—see FIG. 3). To connect a device, a user may be prompted via a GUI to provide input, such as user login or authentication credentials (see, e.g., FIG. 7—Fitbit “Sign In”). Further, as described above, each connection to a device may be set-up as a persistent connection which is managed and maintained to collect a user's health data. Data may be collected over each persistent connection periodically or in response to a trigger event (e.g., a data synchronization event or schedule). In some implementations, a GUI may be provided to enable a user to trigger the synchronization and collection of their health data from specific devices or all connected devices (see, e.g., FIG. 9). An API specific to each connected device may be used to support the connection and communication of data. APIs may be provided as part of an API gateway and/or installed locally on each user's device. In addition, the connections may be supported by suitable hardware, software, networks and other means, including one or more buffers, databases, memories, processors, network interfaces, private data networks, virtual private networks, Wi-Fi networks, LAN or WAN networks, Ethernet cables, coaxial cables, twisted pair cables, fiber optic networks, public switched telephone networks, wireless cellular networks, BLUETOOTH™, BLUETOOTH LE™ (BLE), Wi-Fi, near field communications (NFC), and/or other components to enable the connection and exchange of data. See, e.g., FIGS. 2 and 3 and the components thereof.

As part of step 420, the at least one processor may store prioritization information for the connected devices (e.g., a first prioritization for the connected devices). By way of example, the prioritization information may be stored securely by main server 310 as a set of values or as part of a table. As described above, the prioritization for the connected devices may define an order for collecting health data from the connected devices. Further, the prioritization information for the connected devices may be based on user input received through one or more graphical user interfaces. The user input may include the selection of devices for creating the persistent connections. In some implementations, the order in which devices are connected defines the prioritization order for the connected devices. The user input may also include a device prioritization to change or update the prioritization of the connected devices (see, e.g., FIG. 8). As changes are made by the user to the prioritization for the devices, the stored prioritization information for the connected devices may likewise be updated. For example, in response to user input received through the at least one graphical user interface, a second or updated prioritization for the connected devices may be stored that is different from a first or earlier stored prioritization for the connected devices.

At step 430, the at least one processor may store prioritization information for the data (e.g., a first prioritization for the data). Similar to the prioritization information for connected devices, the prioritization information for data may be stored securely by main server 310 as a set of values or as part of a table. As discussed above, the prioritization for the data may define an order among the data from the connected devices or manually entered data for selecting and applying one or more health data parameters. For example, the prioritization information may be used by main server 310 to control which health data parameters are selected and applied by health engine 360 for calculating a health value or providing a health or wellness recommendation to a user. Through user input entered via one or more GUIs, a user may prioritize a manually entered health data parameter over the same data parameter from one of his or her connected devices or prioritize the parameter from a specific connected device over any other source for that parameter. If the prioritized parameter is unavailable or unobtainable, then main server 310 may use the next prioritized or available source for that parameter in its place.

As described above, the user input from a user may include a data prioritization to change or update the prioritization of the data (see, e.g., FIG. 11). As changes are made by the user to the prioritization for the data, the stored prioritization information for the data (including one or more health data parameters) may likewise be updated. For example, in response to user input received through the at least one graphical user interface, a second or updated prioritization for the data may be stored that is different from a first or earlier stored prioritization for the data. The second or updated prioritization may define an order among the data from the connected devices or manually entered data for selecting and applying one or more health data parameters

At step 440, the at least one processor may receive health data form the devices according to the prioritization for the devices. For example, the at least one processor may collect and store health data periodically from the one or more connected devices in the order defined by the prioritization for the connected devices. As part of step 440, health data (including one or more health data parameters) may by manually entered by a user and stored with other collected data. All health data may be stored with a date stamp and source indicator so that the at least one processor can identify the most current set of health data parameters and the source of each data parameter, as well as determine when data is outdated. Further, in some implementations, the at least one processor may collect the health data in response to a triggering event. For example, the triggering event can be a predetermined time interval (e.g., five minutes, one hour, etc.) between data collections or synchronizations, when the user logs into or an application, and/or when health data of a user is determined to have missing or outdated health data parameters. In some embodiments, the triggering event is defined based on a condition, event, or schedule. By way of example, the triggering event may be defined based on: a period of time since the last collection of health data, a request for a health value, a request for a health or wellness recommendation, an authentication of the user, a connection or reconnection of a device, a data synchronization event or schedule, and/or an expiry of one or more health data parameters.

By way of example, the health data received from the connected devices or manually entered by a user may include user data and health data parameters including one or more of: one or more of age, gender, ethnicity, location, quality of life measures, dietary intake and preferences, weight, height, arm circumference, waist circumference, systolic blood pressure, diastolic blood pressure, total blood pressure, high-density lipoprotein (HDL) cholesterol, low-density lipoprotein (LDL) cholesterol, total cholesterol, oxidized LDL (oxLDL) levels, triglyceride levels, plasma glucose levels, plasma insulin or c-peptide levels, non-esterified fatty acids (NEFA) levels, blood pressure, activity level, moderate muscle strength, vigorous muscle strength, walking distance per time unit, body fat percentage, squat strength, pushup strength, knee pushup strength, plank strength, balance tests, Short Physical Performance Battery (SPPB) score, sleep patterns, blood oxygen saturation (SpO2) level, maximum rate of oxygen (VO2), physical activity patterns, calorie consumption, resting heart rate, breathing rate, respiratory rate, health diseases, family history of health diseases, pregnancy, breast feeding patterns, pregnancy complications, contraceptive practices, perceived stress level, immune markers, vitamin and mineral levels, food intolerance, allergies or allergen markers, microbiome composition and markers, and genotypic or epigenetic information.

As will be appreciated, health data may be updated or revised from time to time (e.g., where there are changes to one or more health data parameters of a user). These updates may be the result of monitoring by one or more of the user's fitness trackers 350 and/or other devices or sources of health data. For example, the health data of a user may change because of an intervention and/or due to the user following one or more health or wellness recommendations (e.g., a diet or exercise plan). In addition, a user's health data may become outdated or incomplete. In some embodiments, main server 310 may take steps to acquire or update the data. For example, a synchronization of the data may be triggered automatically due to a condition, event, or schedule, as described above. Main server 310 may also initiate a data synchronization following a notification to and acceptance by a user. Additionally, or alternatively, the user may be prompted through a GUI or graphical display to check and reconnect devices and/or manually enter one or more health data parameters that are determined to be missing or outdated. Further, main server 310 may initiate attempts to reconnect and/or poll a connected device to collect health data. These and other operations may result in one or more steps of the example process of FIG. 4 being repeated.

At step 450, the at least one processor may calculate a health value. To calculate a health value, such as a biological age or metabolic scale, main server 310 may select and apply one or more of the stored health data parameters to health engine 360 in accordance with the prioritization for the data. For example, using the stored prioritization information, health data parameters may be selected and applied according to their highest ordered source. If the highest ordered health data parameter is outdated or unavailable, then main server 310 applies the second or next highest ordered health data parameter for the calculation, and so on. The process may be repeated for each health data parameter that is required for calculating the health value. If a specific health data parameter is unavailable or missing, the main server 310 may notify the user and/or prompt the user to enter a value for the missing health data parameter.

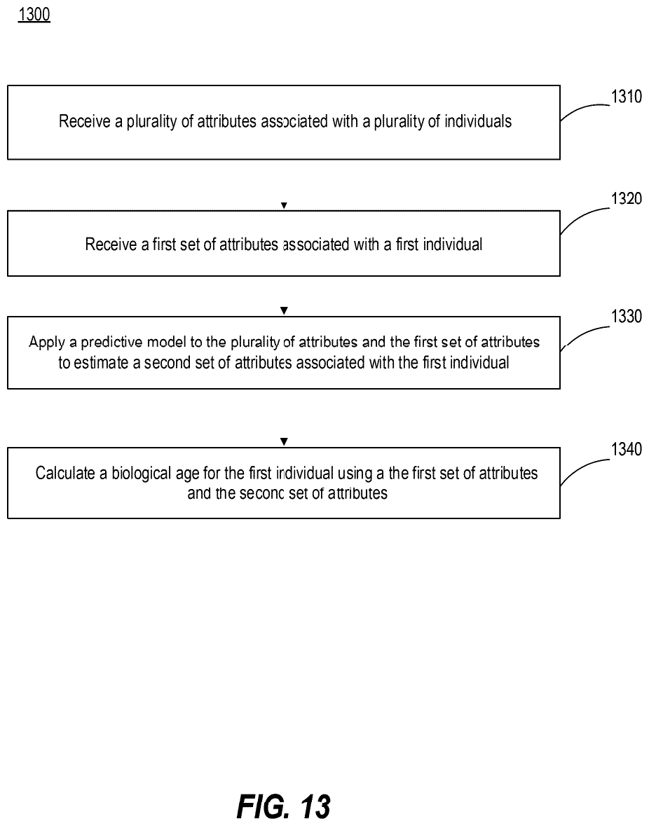

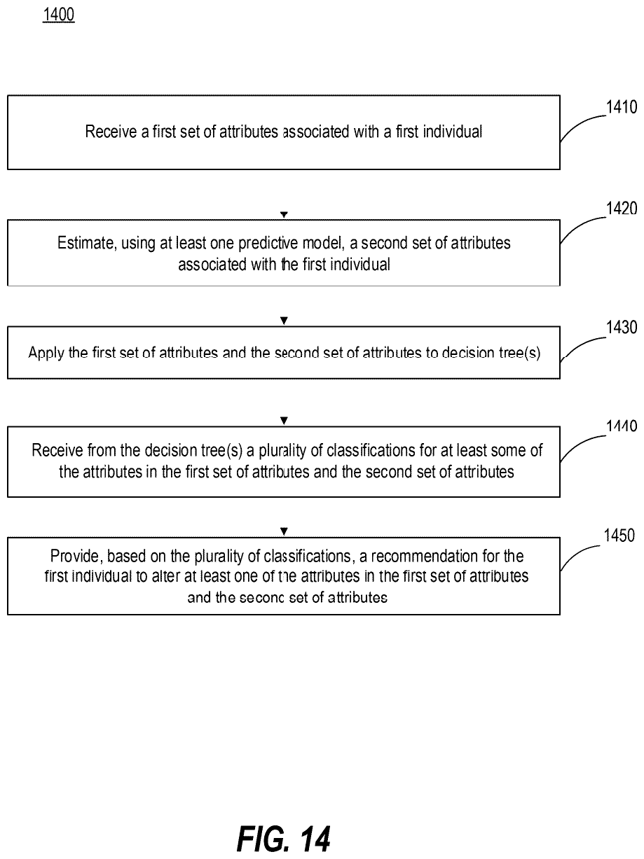

An example method of calculating a biological age is described below with reference to FIG. 13. As part of step 450 or another step, the at least one processor may also generate and provide a health or wellness recommendation. In some embodiments, main server 310 may select and apply one or more health data parameters to health engine 360 to generate a health or wellness recommendation. An example method for calculating a recommendation is described below with reference to FIG. 14.

As described above, one or more health data parameters may be applied to generate a health or wellness recommendation for a user. The parameters may be applied in accordance with the prioritization for the data. In some embodiments, the one or more health data parameters are provided to health engine 360 (FIG. 3) to generate a health or wellness recommendation for the user by using a programmed model, decision tree, and/or one or more machine learning models. The health or wellness recommendation may include, for example, a nutrient recommendation, meal composition recommendation, meal timing commendation, physical activity recommendation, mental activity recommendation, sleep recommendation, health supplement recommendation, health article recommendation, or daily task recommendation. As will be appreciated, other health or wellness recommendations may be provided.

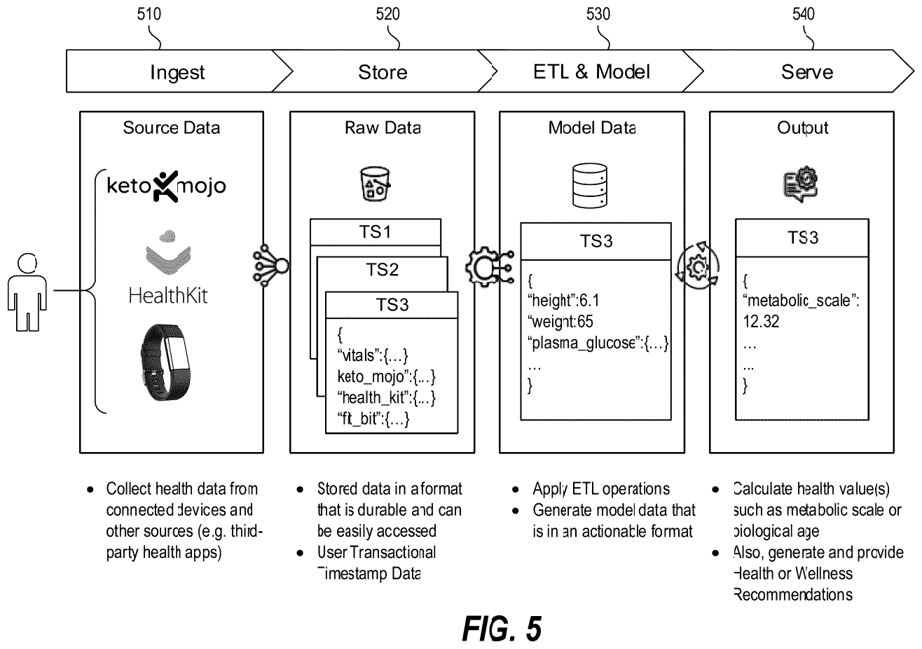

FIG. 5 illustrates an example data ingest and processing method, consistent with embodiments of the present disclosure. The example method 500 may be used to collect and process health data to calculate health values (e.g., biological age or metabolic scale) and/or personalized health or wellness recommendations for a user. The example method may be implemented using one or more processors (e.g., processor(s) 230 of the computing device of FIG. 2) or non-transitory computer readable medium, such as a CPU, FPGA, ASIC, or any other processing structure(s) or storage medium. In some embodiments, example method 500 may be performed by one or more processor(s) of system 300 of FIG. 3, such as user devices 330, main server 310, health engine 360, and/or database manager 370. It is to be understood that the steps shown in FIG. 5 are exemplary only and are not intended to be exhaustive. The steps shown in FIG. 5 may be modified, rearranged, or combined with other steps, depending on the specific application or purpose.

At step 510, the at least one processor may ingest health data from one or more connected sources, such as fitness tracking devices and/or health applications. For example, as shown in FIG. 5, the connected devices include Keto-Mojo, Apple HealthKit, and a wearable fitness tracker (e.g., Fitbit or Garmin). As disclosed herein, the health data may include one or more health data parameters (e.g., weight, heart rate, blood pressure, etc.) and other related data like such as user ID, user name, timestamp (e.g., date and time), and so on. The ingest or collection of data may be performed and repeated, such as when updating or synchronizing data.

At step 520, the at least one processor may store the collected health data in raw form (i.e., raw data). The data may be stored by database manager 370 in a format that is durable and easily accessible. The data may be stored securely and together in a single file or separately under multiple files. The files may be indexed or arranged by user, source and/or date/time (e.g., timestamp data). In some embodiments, the data is stored and organized using user transactional timestamp data. Other types of timestamp data may be employed.

At step 530, the at least one processor may apply one or more extract, transform, load (ETL) operations to obtain health data parameters from the health data. For example, health data parameters may include user's weight, heart rate, blood pressure, blood oxygen saturation level, sleeping patterns, etc. As part of step 530, the obtained health data parameters may be applied to generate model data that is in actionable format for calculating health value(s) and/or recommendations. By way of example, the model data may be used with one or more decision trees or machine learning models.

At step 540, the at least one processor may calculate a health value for the user, based on the health data parameters and/or model data. A health value may be a biological age or a metabolic scale calculated for a user. Health engine 360 may use one or more programmed models, decision trees, and/or machine-learning models (e.g., one or more predictive models) to calculate health value and generate health or wellness recommendations.

Various methods or processes may be implemented for calculating a biological age of a user. One example embodiment is shown in FIG. 13. By way of example, personalized scores may be calculated using received and/or estimated attributes (based on health data parameters), along with weighting coefficients. Other methods or processes may be used, however. Additionally, the at least one processor may provide a health or wellness recommendation, based on the health data parameters of a user. Various methods or processes may be implemented for generating a recommendation for a user, such as the example embodiment described below with reference to FIG. 14. By way of example, the at least one processor may determine a recommendation based on output(s) of a decision tree. The at least one processor may apply different weighting factors to different branches (e.g., attribute-related branches may have a different weighting coefficients), by applying a prioritization order to its branches (e.g., attribute-related branches may be ranked based on an attribute prioritization order for a particular individual), by following further predetermined rules or logic (e.g., using specific decision trees in view of prioritized attributes), or by performing another suitable process based on the particular context or application.

FIGS. 6-12 illustrate example graphical user interfaces for a health data collection and management, consistent with embodiments of the present disclosure. The example graphical user interfaces may be generated and presented with the aid of at least one processor (e.g., processor(s) 230 of the computing device of FIG. 2) or non-transitory computer readable medium, such as a CPU, FPGA, ASIC, or any other processing structure(s) or storage medium. The example graphical user interfaces may be displayed to a user via a display screen of a user device (e.g., a mobile phone, a tablet, a laptop, a computer, a smartwatch, etc.) or other suitable I/O component thereof. It is to be understood that the graphical user interfaces shown in FIGS. 6-12 are exemplary only and are not intended to be exhaustive. Modifications may be made to the example graphical user interfaces shown in FIGS. 6-12 and/or other graphical user interfaces may be generated and presented to a user, depending on the specific application or purpose.

FIG. 6 illustrates an example graphical user interface 600 for connecting and prioritizing devices, consistent with embodiments of the present disclosure. Through interface 600, a user may select the devices to be connected to the system. As shown in FIG. 6, interface 600 may provide a list of connected devices and a list of devices that can be connected. To connect a device, a user may click on it in the list of devices that are available to connect. The at least one processor may then initiate the connection to the device. To establish the connection, the user may be prompted to sign or log in with their credentials to authenticate the access to the device. An example graphical user interface for a user to sign in is described below with reference to FIG. 7. With interface 600, the user may also be given options to, for example, prioritize the connected devices (by clicking “Prioritization My Connections”) and manage and prioritize their health data (by clicking “Manage My Health Data”). Clicking these options may cause further graphical user interfaces to be presented to the user (see, e.g., FIGS. 8-11).

FIG. 7 illustrates an example graphical user interface 700 for user sign in and authentication, consistent with embodiments of the present disclosure. For enabling access and the collection of data from a connected device, a user may be prompted to sign or log in with their credentials. As shown in FIG. 7, interface 700 includes a sign in screen for a user to enter their credentials to sign in for their Fitbit device. The screen also shows an icon, “Continue with Google,” to allow a user to optionally sign in using their Google account. As will be appreciated, other authentication and log in procedures may be implemented.

FIG. 8 illustrates an example graphical user interface 800 for viewing and updating a prioritization for connected devices, consistent with embodiments of the present disclosure. As shown in FIG. 8, interface 800 displays a list of connected devices and allows the user to drag and drop icons of the connected device to establish a prioritization for the devices. In the example of FIG. 8, Apple Health is set at a higher prioritization before Fitbit. A user may also select “save” on interface 800 and the prioritization order will be stored.

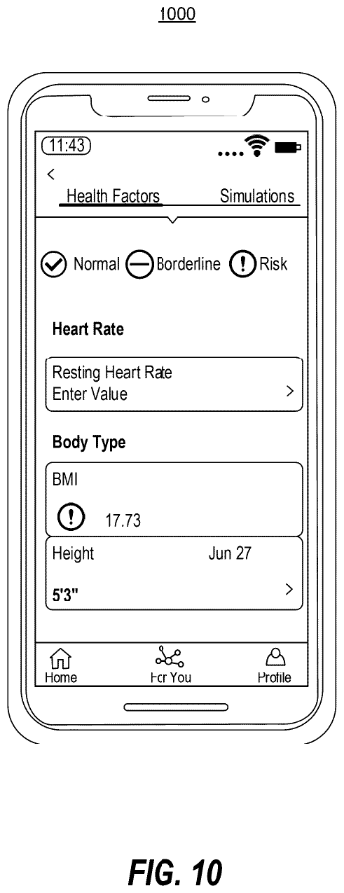

FIG. 9 illustrates an example graphical user interface 900 for synchronizing and managing data, consistent with embodiments of the present disclosure. As shown in FIG. 9, interface 900 displays a list of connected devices (e.g., Keto-Mojo, Apple Health, and Fitbit). A user may input selections among these connected devices to synchronize and collect data from one or more of the devices. Interface 900 also provides a user with an option to manage their health data (by clicking on a “Manage Your Data” button). Selecting this option may cause one or more additional graphical user interfaces to be displayed (see, e.g., FIGS. 10 and 11) to allow a user to manually enter data value and define a prioritization for one or more health data parameters.

FIG. 10 illustrates example graphical user interface 1000 for viewing health data parameters of a user, consistent with embodiments of the present disclosure. The health data parameters may include, for example, anthropometric measurements, age, biological gender, ethnicity, user-defined goals or areas of interest, behaviors, functional tests, medical and laboratory test results, blood levels, food intake, fitness tracker data, genetic profiles, microbiome profiles, etc. As shown in FIG. 10, the interface 1000 shows health data parameters such as heart rate, BMI, and height. Also, on top of the interface 1000 shows different levels and related symbols for grading a health data parameter. For example, the interface 1000 show a user's BMI level as being at “risk” and thus a health data parameter that should be improved upon.

FIG. 11 illustrates an example graphical user interface 1100 for entering one or more health data parameters and managing a prioritization for data, consistent with embodiments of the present disclosure. As shown in FIG. 11, a user may be prompted to manually input data through interface 1100 for one or more health data parameters (such as a user's weight). Non-limiting examples of health data parameters for user input include biological gender, height, weight, waist circumference, arm circumference, ethnicity, and chronological age. It will be appreciated that the exemplary health data parameters for user input presented herein are illustrative only and are not intended to be exhaustive. Other health data parameters for user input may be supported depending on the particular application or context. As further shown in FIG. 11, interface 11 also includes the option for a user to prioritize the data. For example, for the user's weight, this health data parameter may be prioritized as between Apple Health and the user's manually entered weight value. As described herein, the prioritization for the data may define an order for selecting and applying health data parameters among the health data received from the connected device(s) and manually entered health data received from the user through the at least one graphical user interface. In FIG. 11, Apple Health is selected as a first priority source for the user's weight over the manually entered value for the user's weight. Alternatively, the user could select to prioritize the manually entered value over the value provided by Apple Health.

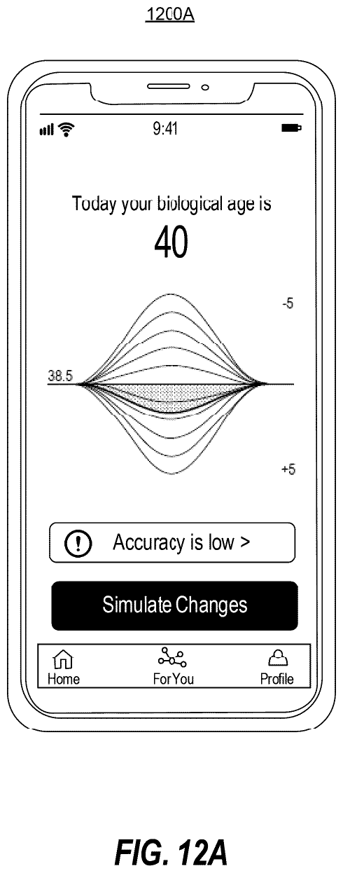

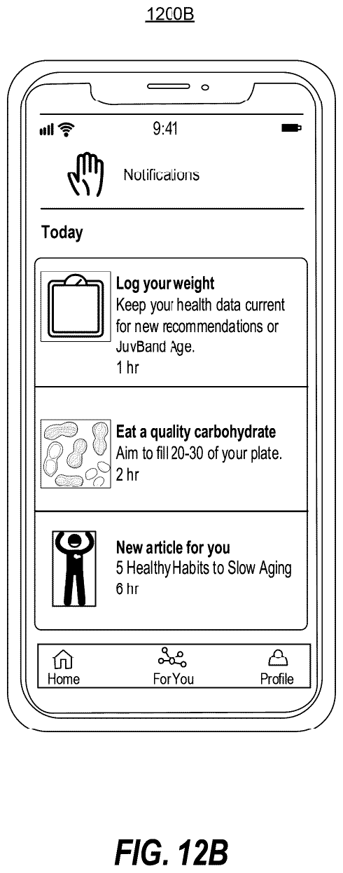

FIGS. 12A and 12B illustrate examples of graphical user interfaces 1200A and 1200B for providing a user with a personalized health value(s) and notifications including personalized health or wellness recommendations. In FIG. 12A, a graphical user interface 1200A is shown in which a calculated health value of a user is presented. In this example, the calculated health value is a biological age of the user. Other health values and interfaces may be used to present results to the user. Furthermore, as shown in FIG. 12B, another graphical user interface 1200B may be provided to present notifications, articles, and one or more health or wellness recommendations to the user. The health or wellness recommendations may be personalized for the user and include recommendations such as dietary patterns and/or physical activities. It will be appreciated that other types of health or wellness recommendations may be determined and provided to a user, and the examples presented herein are illustrative only.

FIG. 13 illustrates an example method 1300 for calculating a biological age for a user, consistent with embodiments of the present disclosure. The example method 1300 may be implemented using one or more processors (e.g., processor(s) 230 of the computing device of FIG. 2) or non-transitory computer readable medium, such as a CPU, FPGA, ASIC, or any other processing structure(s) or storage medium. In some embodiments, example method 1300 may be performed by health engine 360 and/or the other components of FIG. 3. It is to be understood that the steps shown in FIG. 13 are exemplary only and are not intended to be exhaustive. Additional or fewer steps than those shown in FIG. 13 may be performed, depending on the specific application or purpose.

At step 1310, the at least one processor may receive a plurality of attributes associated with a plurality of individuals. An “attribute,” as used with respect to FIG. 13, may refer broadly to any characteristic associated with one or more individuals. By way of example, attributes may be based on collected health data of a user and comprise one or more of metabolic, cardiovascular, muscle, and/or immunity attributes. Non-limiting examples of attributes include anthropometric measurements (e.g., height, weight, waist circumference, arm circumference, etc.), age, biological gender, race, ethnicity, functional tests, medical and laboratory test results, blood levels, heart rate, food intake, sleep schedule, workout schedule, fitness tracker data, genetic profiles, and microbiome profiles, behaviors, and user-defined goals or areas of interest, among others. For example, an attribute may comprise one or more of the following: age, gender, ethnicity, location, quality of life measures, dietary intake and preferences, weight, height, arm circumference, waist circumference, systolic blood pressure, diastolic blood pressure, total blood pressure, high-density lipoprotein (HDL) cholesterol, low-density lipoprotein (LDL) cholesterol, total cholesterol, oxidized LDL (oxLDL) levels, triglyceride levels, plasma glucose levels, plasma insulin or c-peptide levels, non-esterified fatty acids (NEFA) levels, blood pressure, activity level, moderate muscle strength, vigorous muscle strength, walking distance per time unit, body fat percentage, squat strength, pushup strength, knee pushup strength, plank strength, balance tests, Short Physical Performance Battery (SPPB) score, sleep patterns, blood oxygen saturation (SpO2) level, maximum rate of oxygen (VO2), physical activity patterns, calorie consumption, resting heart rate, breathing rate, respiratory rate, health diseases, family history of health diseases, pregnancy, breast feeding patterns, pregnancy complications, contraceptive practices, perceived stress level, immune markers, vitamin and mineral levels, food intolerance, allergies or allergen markers, microbiome composition and markers, and genotypic or epigenetic information. It will be appreciated that the above attributes are merely examples and that the attributes may relate to any combination of attributes from one or more categories, including metabolic, cardiovascular, muscle, and/or immunity attributes. Further, in some embodiments, an attribute may be determined from one or more affirmative or negative responses to one or more health-related questions (e.g., “what is your age” or “what is your weight” or “are you a smoker?” or “are you pregnant?”). In this manner, attributes may be received from each individual through a questionnaire or other list of questions displayed via a health management software application (“app”) or program running on, e.g., a user's mobile phone, laptop, tablet, computer, etc. Attribute data may also be received through online questionnaires managed through one or more websites or through studies or surveys organized by a clinic, health care provider, or organization. Attribute data may also be electronically stored and received from a database or memory device. In some embodiments, attributes are stored as part of enrollment data, survey data, population data, and/or personal health records. Attribute data may also be corrected, revised, or estimated (e.g., in case of incomplete or incorrect entry of such data) and may also be updated (e.g., in case of changes to one or more user attributes due to monitoring (such as by a fitness tracker or other device) or intervention and/or the following of health recommendations by a user and the improvement to their attributes). The above examples are provided for illustration purposes only and are not intended to be exhaustive.

By way of further example, the plurality of attributes associated with the plurality of individuals may be received through various suitable means, including through any electrical medium such as one or more signals, instructions, API calls, databases, memories, hard drives, private data networks, virtual private networks, Wi-Fi networks, LAN or WAN networks, Ethernet cables, coaxial cables, twisted pair cables, fiber optics, public switched telephone networks, wireless cellular networks, BLUETOOTH™, BLUETOOTH LE™ (BLE), Wi-Fi, near field communications (NFC), or any other suitable communication method that provides a medium for exchanging data. For example, the at least one processor may receive the plurality of attributes from one or more databases such as the databases shown in FIG. 3 in communication with database manager 370.

At step 1320, the at least one processor may receive a first set of attributes associated with a particular individual (e.g., a first individual or user). The first set of attributes of the first or particular individual may include the same or similar attributes as those listed above for the plurality of individuals. For example, the first set of attributes may include one or more attributes related to metabolic health, cardiovascular health, weight management, muscle health, immune health and inflammation, weight management, mobility and joint health, liver health, mental and emotional health and performance, stress management, gastrointestinal health, vision, appearance, sleep, biorhythm, men's health, women's health, sexual health and function, nutrient intake, quality of life measures, and/or other health-related attributes. Further, the first set of attributes of the first individual may also be received in the same or similar manner as the plurality of attributes associated with a plurality of individuals. In some embodiments, the first set of attributes may not include all needed or preferred attributes for generating health components or recommendations. That is, the first set of attributes of the first individual may include missing or unknown attributes. Further, following the example process of FIG. 13, step 1320 and the other steps of FIG. 13 (described below) may be repeated when attributes of an individual are corrected or revised (e.g., in case of incomplete or incorrect entry of such data) or updated (e.g., where there are changes to one or more user attributes) due to monitoring (such as by a fitness tracker or other device) or due to intervention and/or the following of health recommendations by a user resulting in the improvement of their attributes).

At step 1330, the at least one processor may apply at least one predictive model to the plurality of attributes and the first set of attributes to estimate a second set of attributes associated with the first individual. The second set of attributes may provide estimates of missing or unknown attributes of the first individual (i.e., attributes that are needed or prescribed, but were not included as part of the first set of attributes for the first individual). Various types of predictive models may be used to generate the second set of attributes, such as a Bayesian network, a Principal Component Analysis (PCA) model, a decision tree, a random forest classifier, a binary classifier, a multiclass classifier, a linear classifier, a neural network, a deep neural network, a support vector machine, a Hidden Markov model, or any other model. The one or more predictive models may also be implemented using non-machine learning algorithms, such as a nearest neighbor model, a regression model, a clustering model, an outliers model, a classification model, a least square fitting model, a time series model, or any other model. In some embodiments, two or more types of models (e.g., two machine-learning models, two non-machine learning models, or a combination of machine-learning and non-machine learning models) may be used in combination with one another, as will be appreciated by those having ordinary skill in the art upon reviewing the present disclosure.

For example, the one or more predictive models may include one or more score models and/or generative models. As a further example, the one or more predictive models may include one or more principal component analysis models. As a still further example, the one or more predictive models may include one or more Bayesian networks. In some embodiments, generative model(s) may be used to generate a synthetic or artificial group of individuals (e.g., a peer group based on population data) that match or correspond to a particular individual. The generative model(s) may also estimate the uncertainty for the individual participant's missing health data. One or more Bayesian networks or statistical models can generate a significantly large set of samples for any kind of individual and closely replicate the distribution of true data. In some embodiments, score model(s) may also be used to generate scores for a particular individual's data. Principal component model(s) or similar models may apply weighted ranks for health components, e.g., from healthy to poor metabolism on a continuous scale. Peer group scores may be generated that show an individual's relation to their peer group in relation to metabolism and/or other health components. Such scoring may be expressed in terms of a scale (e.g., above average, average, below average) or as a unit of time (e.g., years relative to the individual's chronological age). As further disclosed herein, guideline scores may also be generated to show an individual's health components relative to guideline or predetermined thresholds for their chronological age. These scores may also be expressed and displayed to a user relative to a scale or unit of time. The thresholds may be derived from one or more sources of expert guidelines or recommendations, such as leading sources of health guidelines or recommendations like the National Institutes of Health (NIH) or World Health Organization (WHO).

By way of further example, one or more Bayesian networks (or other statistical models) may be applied to the plurality of attributes associated with the plurality of individuals to generate a plurality of artificial attributes associated with a plurality of artificial individuals. In some embodiments, the artificial individuals may be generated or identified so as to match the first individual based on the first set of attributes, such as through the use of threshold(s) or other suitable means related to one or more attributes (e.g., age, gender, ethnicity, etc). A collection of such one or more artificial attributes generated according to the processes described herein may be associated with the synthetic or artificial individuals. With the matching group of artificial individuals and attributes, the predictive model(s) may estimate missing or unknown attributes of the first individual, so as to generate a second set of attributes associated with the first individual. In some embodiments, the generated second set of attributes may be larger than the first set of attributes. Further, in some embodiments, the predictive model(s) may output a confidence score associated with the generated artificial attributes and/or second set of attributes.

Consistent with FIG. 13 and the other examples embodiments disclosed herein, the plurality of attributes of the plurality of individuals, the first set of attributes of the particular individual, and the estimated second set of attributes may include any combination of attributes for one or more categories, including metabolic health, cardiovascular health, weight management, muscle health, immune health and inflammation, weight management, mobility and joint health, liver health, mental and emotional health and performance, stress management, gastrointestinal health, vision, appearance, sleep, biorhythm, men's health, women's health, sexual health and function, nutrient intake, quality of life measures, and/or other health-related attributes. Examples of attributes are provided in the preceding paragraphs.

At step 1340, the at least one processor may calculate a biological age for the first individual using the first set of attributes and the second set of attributes. Various suitable processes or formulas for calculating a biological age may be used. For example, the following formula may be applied to an individual's attributes to calculate a biological age:

BA=CA+a*S₁+b*S₂+ . . . n*Sₙ

In the formula above, BA may correspond to the particular individual's biological age, CA may correspond to the particular individual's chronological age, S₁ may correspond to a score associated with a first attribute, a may correspond to a weighting coefficient associated with the first attribute, S₂ may correspond to a score associated with a second attribute, b may correspond to a weighting coefficient associated with the second attribute, Sₙ may correspond to a score associated with an n^{th} attribute, n may correspond to a weighting coefficient associated with the n^{th} attribute, and so forth. Each score (S₁, S₂, Sₙ, etc.) may in turn be calculated using a formula associated with one or more attributes. In some embodiments, for example, a score may be calculated using one or more medical guidelines. As a non-limiting example, in embodiments where body mass index (BMI) is an attribute, a score may correspond to a difference between a particular individual's body mass index and an ideal body mass index according to a medical guideline. Accordingly, the particular individual's score may lead to a lower biological age when it is closer to the ideal body mass index, and it may lead to a higher biological age when it is further from the ideal body mass index. Other methods of calculating a score may be used, however, which may include using mean values, standard deviations, best fit models, least square fitting, regressions, statistical models, machine-learning models, or any other process of assessing an individual's attribute.

As a further example, in embodiments where a plurality of artificial attributes associated with a plurality of artificial individuals are generated, a range of biological ages may be calculated for at least some of the artificial individuals. The plurality of artificial individuals may be generated so as to match the particular individual, such as through the use of a threshold applied to at least some of the particular individual's attributes, as discussed above. The same or similar formula as described above may be used to calculate the range of biological ages, although other processes may be used. The calculated range of biological ages may be used to calculate the biological age of the particular individual, such as by estimating or refining the biological age. For example, the particular individual's biological age may be estimated as the mean biological age in the range of biological ages, or a calculated biological age may be increased or decreased by a predetermined amount based on deviations between the calculated biological age and the range of biological ages. Other process for calculating a biological age using artificial attributes may be used, as will be appreciated by those having ordinary skill in the art upon reviewing the present disclosure.

The example method 1300 of FIG. 13 may be repeated or otherwise executed to provide an updated biological age for an individual. For example, the method of FIG. 13 may be performed when one or more first attributes of an individual are corrected or revised (e.g., in case of incomplete or incorrect entry of such data) or updated (e.g., where there are changes to one or more user attributes) due to monitoring (such as by a fitness tracker or other device) or due to intervention and/or the following of health recommendations by a user resulting in the improvement to at least one of their attributes). The entry of new or updated attribute(s) for an individual may automatically trigger the execution of the method of FIG. 13 and calculation of an individual's biological age. Additionally, or alternatively, an individual may manually instruct (e.g., through at least one graphical user interface and/or user input entered with their user device) the execution of the method of FIG. 13 to see how their biological age changes as a result of an improvement in one or more of their health parameters or attributes.

Consistent with the present disclosure, the biological age calculated by method 1300 may be based on a plurality of attributes, including attributes related to metabolic health, cardiovascular health, weight management, muscle health, immune health and inflammation, weight management, mobility and joint health, liver health, mental and emotional health and performance, stress management, gastrointestinal health, vision, appearance, sleep, biorhythm, men's health, women's health, sexual health and function, nutrient intake, quality of life measures, and/or other health-related attributes. Further, weighting factors may be applied to one or more attributes so that some attribute data weighs more heavily than other attribute data when calculating an individual's biological age. Such weighting factors may be selected for an individual based on their personal attributes and/or needed areas of improvement. In some embodiments, weighting factors may vary across one or more attributes or attribute categories (e.g., metabolic, cardiovascular, muscle. and/or immunity, etc.). A user may also select which attribute(s) to focus on (i.e., weigh more heavily) and/or they may select to have their biological age calculated based on one or more specific categories of attributes (e.g., a metabolic biological age, a cardiovascular biological age, a muscle biological age, or an immunity biological age alone or a biological age based on a combination of different categories of attributes, such as metabolic and cardiovascular attributes). As will be appreciated, these are just examples and other weighting arrangements and features may be implemented, consistent with the present disclosure.

In some embodiments, a biological age for a user may be calculated using one or more specified or target attributes. For example, the at least one processor may be configured to receive at least one hypothetical or target attribute for a particular individual. The hypothetical attribute(s) may correspond to a target goal or scenario planning aimed at improving one or more attributes for the particular individual. Using the hypothetical attribute(s) alone or together with existing attributes of the user, the at least one processor may be configured to recalculate the biological age. Accordingly, the particular individual may be able to see the effect of improving specific attribute(s) on his or her biological age and may consequently be motivated to alter his or her behaviors, including related to diet and/or exercise.

In some embodiments, the at least one processor may be configured to provide trend modeling and allow a user to see their biological age in the future, assuming certain attribute(s) and the maintenance of a current lifestyle of diet, exercise, etc. Trend modeling may be implemented using aging data or similar surveys tracking the attributes of a population set (i.e., plurality of users) over time in view of their lifestyle. Examples of such aging data or surveys include The Irish LongituDinal Study on Aging (TILDA) conducted by Trinity College Dublin, which is a large-scale, longitudinal study on aging in Ireland. Such datasets may be used for modeling and group matching to predict a user's biological age in the future. In such embodiments, the at least one processor may receive a set of input(s) including one or more current attributes of a user and lifestyle data for the user (e.g., defining the user's lifestyle habits of diet, exercise, drinking, smoking, etc). Hypothetical attributes and/or a further target date may also be provided as input data. With these inputs, the at least one processor may calculate a future biological age for the user. In this manner, the user may be able to see the impact of attributes and lifestyle behavior on their future biological age and be motivated to seek health recommendations and intervention to improve their aging profile and overall health.

Furthermore, in some embodiments, the at least one processor may be configured to provide health or wellness recommendations. The recommendations may be provided to positively impact one or more aspects of an individual's health, including a biological age, one or more attributes, mental or emotional health, or any other aspect of a user's health or wellness. In some embodiments, machine learning models may be used to learn the impact of following a recommendation for a particular individual. For example, a machine learning model may learn the impact of a meal plan, exercise schedule, sleep patterns, supplement regime, or any other recommendation, and may consequently tailor recommendations for a particular user or individual, as disclosed herein. It will be appreciated that other approaches for providing and refining recommendations may be used.

FIG. 14 illustrates an example method 1400 for providing a personalized recommendation, consistent with embodiments of the present disclosure. The example method 1400 may be implemented using at least one processor (e.g., processor(s) 230 of the computing device of FIG. 2) or non-transitory computer readable medium, such as a CPU, FPGA, ASIC, or any other processing structure(s) or storage medium. In some embodiments, example method 1400 may be performed by health engine 360 and/or the other components of FIG. 3. It is to be understood that the steps shown in FIG. 14 are exemplary only and are not intended to be exhaustive. Additional or fewer steps than those shown in FIG. 14 may be performed, depending on the specific application or purpose.

At step 1410, the at least one processor may receive a first set of attributes associated with a particular individual (e.g., a first individual or user). The first set of attributes may be the same or similar as those listed above (e.g., anthropometric measurements, age, race, biological gender, blood levels, etc.) and may be based on collected health data. Furthermore, the first set of attributes may be received in the same or similar manner as discussed above (e.g., through one or more API calls, databases, memories, networks, etc.).

At step 1420, the at least one processor may estimate, using at least one predictive model, a second set of attributes for the particular individual. The estimated second set of attributes may be used to supplement the first set of attributes and/or address missing attribute data (e.g., one or more attributes related to metabolic health, cardiovascular health, weight management, muscle health, immune health and inflammation, weight management, mobility and joint health, liver health, mental and emotional health and performance, stress management, gastrointestinal health, vision, appearance, sleep, biorhythm, men's health, women's health, sexual health and function, nutrient intake, quality of life measures or other health-related attributes). The same or similar predictive model(s) as those described above may be used to estimate the second set of attributes (e.g., one or more Bayesian networks, Principal Component Analysis (PCA) models, decision trees, random forest classifiers, binary classifiers, multiclass classifiers, linear classifiers, neural networks, deep neural networks, support vector machines, Hidden Markov models, nearest neighbor models, regression models, clustering models, outliers models, classification models, least square fitting models, time series models, and/or any other machine learning or non-machine learning models).

At step 1430, the at least one processor may prioritize one or more of the attributes in the first set of attributes and the second set of attributes. Various suitable prioritization logic or methods may be used. In some embodiments, for example, attributes may be prioritized based on their deviation from a reference value, which may include a medical guideline, a personalized recommended value, a target value, a peer group value, or any other value. Accordingly, an attribute with a higher deviation from the reference value may be deemed higher priority than an attribute with a lower deviation from the reference value. As a further example, attributes may be prioritized manually, such as through an individual's preference, an individual's history (e.g., previous attributes that impacted the individual's health), a user-defined goal, an application manager-defined goal (e.g., a health coach), or any other input. In such embodiments, the attributes may be ranked manually in a preferred order to best suit a particular individual. Although not shown, in some embodiments the at least one processor may display on a user device prioritized attributes to inform a user.

As a further example, a machine learning model may be used to learn an attribute priority or ranking. In such embodiments, attributes and/or other data related to the particular individual and/or other individuals may be used. For instance, an individual's health history over a period of time (e.g., one week, one month, one year, or several years) may be fed to one or more neural networks (e.g., a deep neural network, a convolutional neural network, a recursive neural network, etc.), a random forest, a support vector machine, or any other suitable machine learning model, trained to analyze the effect of attributes on an individual's health. In one embodiment, the neural networks may learn to identify patterns in the particular individual's attributes that lead to the highest impact on the particular individual's health. For instance, the over a period of a year, the neural networks may learn that food or macronutrients impact the particular individual's health with disparate effects and may prioritize the foods or macronutrients accordingly. As another example, the neural networks may learn that a particular combination of attributes impact the particular's health significantly, such as a combination of meal, exercise, and sleep timing. In another embodiment, the neural networks may be trained to classify attribute patterns as either “positive” or “negative” for an individual's health, where the neural networks may be trained using attribute patterns of various individuals reflecting either a positive or a negative change. For example, a first set of training patterns where the effect of one or more attributes may be beneficial may be labeled as “positive,” while a second set of training patterns where the effect of one or more attributes may be detrimental may be labeled as “negative.” Other labeling conventions could be used, however, as will be appreciated by those having ordinary skill in the art. Weights or other parameters of the neural network may be adjusted based on its output with respect to a third, non-labeled set of training patterns based on whether the neural networks predict the outcome of the pattern to be “positive” or “negative,” and the process may be repeated with additional training patterns or with live data. The trained neural networks may be applied to monitor the attribute patterns of a particular individual, so as to arrive at an attribute priority. The examples provided herein for prioritizing attributes are not intended to be exhaustive and are intended to be illustrative only.

At step 1440, the at least one processor may input or apply the first set of attributes and the second set of attributes to at least one decision tree. The decision tree(s) may be the same or similar to those discussed above (e.g., classification decision trees, regression decision trees, linear discriminant classification trees, quadratic discriminant classification trees, logistic regression classification trees, classification and regression trees (CART), multiple additive regression trees (MART), prediction analysis for microarrays (PAM), random forest decision trees, nearest neighbor decision trees, or any other suitable machine learning or non-machine learning decision tree algorithms). In some embodiments, as part of step 1040, one or more decision tree(s) may be selected and applied based on an individual's health interests or the type or range of recommendations selected or needed by an individual (e.g., through smartphone app and/or graphical user interface). Additionally, or alternatively, specific types or categories of decision tree(s) (e.g., decision trees for metabolic health; cardiovascular health; immune system health; muscular or activity health; and so on) may be selected by the at least one processor based on an individual's attributes and identified areas for improvement. By way of example, the prioritized order of attributes for a particular individual determined as part of step 1030 may be used to select and apply one or more specific decision tree(s) (e.g., a metabolic health decision tree. a cardiovascular health decision tree; and so on) to the first and second sets of attributes for generating health recommendations to the individual.

In some embodiments, the at least one processor is configured to provide a health recommendation to improve a prioritized attribute among the first set of attributes and the second set of attributes. Further, the at least one processor may be configured to select at least one of a plurality of decision trees based on the prioritized attribute(s) to provide the health recommendation for the individual, wherein each of the plurality of decision trees relates to a different area of health. Examples of decision trees have been provided herein. To further illustrate, decision trees may be provided for metabolic health, cardiovascular health, weight management, muscle health, immune health, nutrient intake, quality of life measures, male or female-based health, psychological health, and/or gastro-intestinal health. These are non-limiting examples and other types of decision trees may be provided.

At step 1450, the at least one processor may receive from the decision tree(s) a plurality of classifications for the first set of attributes and the second set of attributes. The plurality of classifications may be the same or similar as those described above (e.g., classifications of “low,” “normal,” “high,” “elevated,” and “strongly elevated,” among others).

At step 1460, the at least one processor may provide, based on the plurality of classifications, at least one recommendation to alter the prioritized at least one attribute in the first set of attributes and the second set of attributes. In some embodiments, for example, outputs of the decision tree(s) (or other logic or algorithm) may be weighed differently based on the attribute priority. Following the diet-based example described above, for instance, the at least one processor may arrive at one or more dietary-based recommendations based on a diet-based decision tree's outputs so as to alter a high priority attribute, such as by applying different weighting factors to different branches (e.g., attribute-related branches may have a different weighting coefficients), by applying a prioritization order to its branches (e.g., attribute-related branches may be ranked based on an attribute prioritization order for a particular individual), by following further predetermined rules or logic (e.g., using specific decision trees in view of prioritized attributes), or by performing another suitable process based on the particular context or application. Instead of tallying votes for each type of recommended diet, for example, the at least one processor may weigh particular classifications more strongly based on the attribute priority, such as by weighing “strongly elevated” classifications higher than “elevated” or “low” classifications, although any other priority order may be used as discussed above.

The diagrams and components in the figures described above illustrate the architecture, functionality, and operation of possible implementations of systems, methods, and computer hardware or software products according to various example embodiments of the present disclosure. For example, each block in a flowchart or diagram may represent a module, segment, or portion of code, which includes one or more executable instructions for implementing the specified logical functions. It should also be understood that in some alternative implementations, functions indicated in a block may occur out of order noted in the figures. By way of example, two blocks or steps shown in succession may be executed or implemented substantially concurrently, or two blocks or steps may sometimes be executed in reverse order, depending upon the functionality involved. Furthermore, some blocks or steps may be omitted. It should also be understood that each block or step of the diagrams, and combination of the blocks or steps, may be implemented by special purpose hardware-based systems that perform the specified functions or acts, or by combinations of special purpose hardware and computer instructions. Computer program products (e.g., software or program instructions) may also be implemented based on the described embodiments and illustrated examples.

It should be appreciated that the above-described systems and methods may be varied in many ways and that different features may be combined in different ways. In particular, not all the features shown above in a particular embodiment or implementation are necessary in every embodiment or implementation. Further combinations of the above features and implementations are also considered to be within the scope of the herein disclosed embodiments or implementations.

While certain embodiments and features of implementations have been described and illustrated herein, modifications, substitutions, changes and equivalents will be apparent to those skilled in the art. It is, therefore, to be understood that the appended claims are intended to cover all such modifications and changes that fall within the scope of the disclosed embodiments and features of the illustrated implementations. It should also be understood that the herein described embodiments have been presented by way of example only, not limitation, and various changes in form and details may be made. Any portion of the systems and/or methods described herein may be implemented in any combination, except mutually exclusive combinations. By way of example, the implementations described herein can include various combinations and/or sub-combinations of the functions, components and/or features of the different embodiments described.

Moreover, while illustrative embodiments have been described herein, the scope of the present disclosure includes any and all embodiments having equivalent elements, modifications, omissions, combinations (e.g., of aspects across various embodiments), adaptations or alterations based on the embodiments disclosed herein. Further, elements in the claims are to be interpreted broadly based on the language employed in the claims and not limited to examples described herein or during the prosecution of the present application. Instead, these examples are to be construed as non-exclusive. It is intended, therefore, that the specification and examples herein be considered as exemplary only, with a true scope and spirit being indicated by the following claims and their full scope of equivalents.

What is claimed is:

1. A computer-implemented method for persistent health data collection and multi-level prioritization, the method comprising the following steps performed by at least one processor:
providing at least one graphical user interface to a user that is configured to receive user input for one or more of a device selection, a device prioritization, and a data prioritization;
creating, using application program interfaces (APIs), persistent connections to collect health data from one or more devices, wherein a first prioritization for the connected devices is stored based on user input received through the at least one graphical user interface, the first prioritization for the connected devices defining an order for collecting health data from the connected devices;
storing, based on further user input received through the at least one graphical user interface, a first prioritization for the data, the first prioritization for the data defining an order among the health data from the connected devices for selecting and applying one or more health data parameters;
receiving, using the persistent connections, health data from the devices in accordance with the first prioritization for the devices; and
calculating a health value for the user using one more health data parameters based on the received health data, wherein the health data parameters are selected and applied according to the first prioritization for the data.

2. The method of claim 1, further comprising:
periodically collecting the health data from the one or more connected devices in the order defined by the first prioritization for the connected devices.

3. The method of claim 1, further comprising:
collecting health data in response to a triggering event, the triggering event causing the at least one processor to automatically collect the health data from the one or more connected devices in the order defined by the first prioritization for the connected devices.

4. The method of claim 1, further comprising:
collecting health data in response to a triggering event, the triggering event causing the at least one processor to prompt the user to manually enter health data through the at least one graphical user interface.

5. The method according to claim 3 or 4, wherein the triggering event is defined based on at least one of a condition, event, or schedule.

6. The method according to claim 3 or 4, wherein the triggering event is defined based on at least one of: a period of time since the last collection of health data, a request for a health value, a request for a health or wellness recommendation, an authentication of the user, a connection or reconnection of a device, a data synchronization event or schedule, or an expiry of one or more health data parameters.

7. The method of claim 1, wherein the user input received through the at least one graphical user interface further includes manually entered health data for the user, the manually entered health data including one or more health data parameters.

8. The method of claim 7, wherein the first prioritization for the data defines an order for selecting and applying health data parameters among the health data received from the connected devices and manually entered health data received from the user through the at least one graphical user interface.

9. The method of claim 8, wherein the user input received through the at least one graphical user interface further includes a selection to prioritize one or more health data parameters manually entered by the user.

10. The method of claim 1, wherein the health data includes user data and health data parameters including one or more of age, gender, ethnicity, location, quality of life measures, dietary intake and preferences, weight, height, arm circumference, waist circumference, systolic blood pressure, diastolic blood pressure, total blood pressure, high-density lipoprotein (HDL) cholesterol, low-density lipoprotein (LDL) cholesterol, total cholesterol, oxidized LDL (oxLDL) levels, triglyceride levels, plasma glucose levels, plasma insulin or c-peptide levels, non-esterified fatty acids (NEFA) levels, blood pressure, activity level, moderate muscle strength, vigorous muscle strength, walking distance per time unit, body fat percentage, squat strength, pushup strength, knee pushup strength, plank strength, balance tests, Short Physical Performance Battery (SPPB) score, sleep patterns, blood oxygen saturation (SpO2) level, maximum rate of oxygen (VO2), physical activity patterns, calorie consumption, resting heart rate, breathing rate, respiratory rate, health diseases, family history of health diseases, pregnancy, breast feeding patterns, pregnancy complications, contraceptive practices, perceived stress level, immune markers, vitamin and mineral levels, food intolerance, allergies or allergen markers, microbiome composition and markers, and genotypic or epigenetic information.

11. The method of claim 1, wherein the first prioritization for the connected devices is defined based on an order in which the connections to the devices are established.

12. The method of claim 1, further comprising:
storing a second prioritization for the connected devices in response to further user input received through the at least one graphical user interface, the second prioritization for the connected devices being different from the first prioritization for the connected devices that defines an order for collecting health data from the connected devices.

13. The method of claim 1, further comprising:
storing a second prioritization for the data in response to further user input received through the at least one graphical user interface, the second prioritization for the data being different from the first prioritization for the data that defines an order among the data from the connected devices or manually entered data for selecting and applying one or more health data parameters.

14. The method of claim 1, further comprising:
parsing the received health data to obtain the one or more health data parameters.

15. The method of claim 14, wherein parsing the received health data comprises applying one or more extract, transform, load (ETL) operations to the received health data to obtain the one or more health data parameters.

16. The method of claim 1, further comprising:
applying the one or more health data parameters to generate a health or wellness recommendation for the user.

17. The method of claim 16, further comprising:
applying one or more health data parameters to a health engine that generates a health or wellness recommendations for the user by using at least one of a programmed model, decision tree, or machine learning model.

18. The method of claim 16, wherein the health or wellness recommendation for the user includes at least one of a nutrient recommendation, meal composition recommendation, meal timing commendation, physical activity recommendation, mental activity recommendation, sleep recommendation, health supplement recommendation, health article recommendation, or daily task recommendation.

19. A computer-implemented system for persistent health data collection and multi-level prioritization, the system comprising at least one processor configured to:
provide at least one graphical user interface to a user that is configured to receive user input for one or more of a device selection, a device prioritization, and a data prioritization;
create, using application program interfaces (APIs), persistent connections to collect health data from one or more devices, wherein a first prioritization for the connected devices is stored based on user input received through the at least one graphical user interface, the first prioritization for the connected devices defining an order for collecting health data from the connected devices;
store, based on further user input received through the at least one graphical user interface, a first prioritization for the data, the first prioritization for the data defining an order among the data from the connected devices for selecting and applying one or more health data parameters;
receive, using the persistent connections, health data from the devices in accordance with the first prioritization for the devices; and
calculate a health value for the user using one more health data parameters based on the received health data, wherein the health data parameters are selected and applied according to the first prioritization for the data.

20. The system of claim 19, wherein the at least one processor is further configured to:
periodically collect the health data from the one or more connected devices in the order defined by the first prioritization for the connected devices.

21. The system of claim 19, wherein the at least one processor is further configured to:
collect health data in response to a triggering event, the triggering event causing the at least one processor to automatically collect the health data from the one or more connected devices in the order defined by the first prioritization for the connected devices.

22. The system of claim 19, wherein the at least one processor is further configured to:
collect health data in response to a triggering event, the triggering event causing the at least one processor to prompt the user to manually enter health data through the at least one graphical user interface.

23. The system according to claim 21 or 22, wherein the triggering event is defined based on at least one of a condition, event, or schedule.

24. The system according to claim 21 or 22, wherein the triggering event is defined based on at least one of: a period of time since the last collection of health data, a request for a health value, a request for a health or wellness recommendation, an authentication of the user, a connection or reconnection of a device, a data synchronization event or schedule, or an expiry of one or more health data parameters

25. The system of claim 19, wherein the user input received through the at least one graphical user interface further includes manually entered health data for the user.

26. The system of claim 25, wherein the first prioritization for the data defines an order for selecting and applying health data parameters among the health data received from the connected devices and manually entered health data received from the user through the at least one graphical user interface.

27. The system of claim 26, wherein the user input received through the at least one graphical user interface further includes a selection to prioritize one or more health data parameters manually entered by the user.

28. The system of claim 19, wherein the health data includes user data and health data parameters including one or more of age, gender, ethnicity, location, quality of life measures, dietary intake and preferences, weight, height, arm circumference, waist circumference, systolic blood pressure, diastolic blood pressure, total blood pressure, high-density lipoprotein (HDL) cholesterol, low-density lipoprotein (LDL) cholesterol, total cholesterol, oxidized LDL (oxLDL) levels, triglyceride levels, plasma glucose levels, plasma insulin or c-peptide levels, non-esterified fatty acids (NEFA) levels, blood pressure, activity level, moderate muscle strength, vigorous muscle strength, walking distance per time unit, body fat percentage, squat strength, pushup strength, knee pushup strength, plank strength, balance tests, Short Physical Performance Battery (SPPB) score, sleep patterns, blood oxygen saturation (SpO2) level, maximum rate of oxygen (VO2), physical activity patterns, calorie consumption, resting heart rate, breathing rate, respiratory rate, health diseases, family history of health diseases, pregnancy, breast feeding patterns, pregnancy complications, contraceptive practices, perceived stress level, immune markers, vitamin and mineral levels, food intolerance, allergies or allergen markers, microbiome composition and markers, and genotypic or epigenetic information.

29. The system of claim 19, wherein the first prioritization for the connected devices is defined based on an order in which the connections to the devices are established.

30. The system of claim 19, wherein the at least one processor is further configured to:
store a second prioritization for the connected devices in response to further user input received through the at least one graphical user interface, the second prioritization for the connected devices being different from the first prioritization for the connected devices that defines an order for collecting health data from the connected devices.

31. The system of claim 19, wherein the at least one processor is further configured to:
store a second prioritization for the data in response to further user input received through the at least one graphical user interface, the second prioritization for the data being different from the first prioritization for the data that defines an order among the data from the connected devices or manually entered data for selecting and applying one or more health data parameters.

32. The system of claim 19, wherein the at least one processor is further configured to:
parse the received health data to obtain the one or more health data parameters.

33. The system of claim 32, wherein the at least one processor is further configured to:
apply one or more extract, transform, load (ETL) operations to the received health data to obtain the one or more health data parameters.

34. The system of claim 19, wherein the at least one processor is further configured to:
apply the one or more health data parameters to generate a health or wellness recommendation for the user.

35. The system of claim 34, wherein the at least one processor is further configured to:
apply one or more health data parameters to a health engine that generates a health or wellness recommendations for the user by using at least one of a programmed model, decision tree, or machine learning model.

36. The system of claim 34, wherein the health or wellness recommendation for the user includes at least one of a nutrient recommendation, meal composition recommendation, meal timing commendation, physical activity recommendation, mental activity recommendation, sleep recommendation, health supplement recommendation, health article recommendation, or daily task recommendation.

37. The system of claim 19, wherein the health value comprises a biological age of the user.

38. The system of claim 37, further comprising at least one predictive model configured to calculate the biological age of the user, wherein the predictive model comprises one or more machine learning models.

39. A non-transitory computer readable medium comprising instructions which are executable by at least one hardware processor to perform the following operations:
providing at least one graphical user interface to a user that is configured to receive user input for one or more of a device selection, a device prioritization, and a data prioritization;
creating, using application program interfaces (APIs), persistent connections to collect health data from one or more devices, wherein a first prioritization for the connected devices is stored based on user input received through the at least one graphical user interface, the first prioritization for the connected devices defining an order for collecting health data from the connected devices;
storing, based on further user input received through the at least one graphical user interface, a first prioritization for the data, the first prioritization for the data defining an order among the data from the connected devices for selecting and applying one or more health data parameters;
receiving, using the persistent connections, health data from the devices in accordance with the first prioritization for the devices; and
calculating a health value for the user using one more health data parameters based on the received health data, wherein the health data parameters are selected and applied according to the first prioritization for the data.
